(19) United States
(12) Patent Application Publication
(10) Pub. No.: US 2024/0016996 A1
(43) Pub. Date: Jan. 18, 2024

(54) EXTENDABLE DEPTH DRESSINGS WITH TRANSPARENT CAPABILITY

(71) Applicant: KCI Manufacturing Unlimited Company, Westmeath (IE)

(72) Inventor: Christopher Brian LOCKE, San Antonio, TX (US)

(21) Appl. No.: 18/039,918

(22) PCT Filed: Nov. 16, 2021
(86) PCT No.: PCT/IB2021/060595

§ 371 Date: Jun. 1, 2023

Related U.S. Application Data
(60) Provisional application No. 63/122,341, filed on Dec. 7, 2020.

(51) Int. Cl. A61M 1/00

(52) U.S. Cl. CPC A61M 1/915; A61M 1/918; A61M 1/92

(57) ABSTRACT

An example apparatus for treating a tissue site with negative pressure may include a primary manifold configured to move between a retracted state and an extended state. The primary manifold may include a top surface and a bottom surface positioned opposite the top surface and configured to face toward the tissue site. Further, the primary manifold may include a pleat positioned adjacent to an extension zone that is configured to extend outward from the bottom surface of the primary manifold toward the tissue site when the primary manifold is in the extended state. Also disclosed are other apparatus, dressings, systems, and methods.

### CROSS-REFERENCE TO RELATED APPLICATIONS

This application is a U.S. National Stage entry of International Patent Application No. PCT/IB2021/060595, filed on Nov. 16, 2021, which claims the benefit of priority to U.S. Provisional Application No. 63/122,341, filed on Dec. 7, 2020, which is incorporated herein by reference in its entirety.

### TECHNICAL FIELD

The invention set forth in the appended claims relates generally to tissue treatment systems and more particularly, but without limitation, to dressings for tissue treatment and methods for tissue treatment with negative pressure.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

In some embodiments, a dressing characterized as exhibiting decreased tensile strength, increased flexure, and/or improved conformability with respect to a tissue site may be advantageously employed in the provision of negative-pressure therapy. For example, the increased flexure and/or improved conformability of the dressing may provide for better contact between the tissue site and a tissue site-facing surface of the dressing. The improved contact between the dressing and the tissue site may have the effect of inducing micro-strain across substantially all of the tissue site, whereby cells across the tissue site experience strain, improving the outcome of the negative-pressure therapy.

In some example embodiments, an apparatus for treating a tissue site with negative pressure may include a primary manifold configured to move between a retracted state and an extended state. The primary manifold may include a top surface and a bottom surface positioned opposite the top surface and configured to face toward the tissue site. In some example embodiments, the primary manifold may include a pleat positioned adjacent to an extension zone. The extension zone may be configured to extend outward from the bottom surface of the primary manifold toward the tissue site when the primary manifold is in the extended state. For example, in some embodiments, the bottom surface of the primary manifold may be configured to form a convex shape in conformity with the tissue site when the primary manifold is in the extended state. Further, in some embodiments, the pleat may include a fold or undulation in the primary manifold that is configured to permit portions of the primary manifold to move away from one another when the primary manifold moves from the retracted state to the extended state.

Further, in some example embodiments, a system for treating a tissue site with negative pressure may include the apparatus including the primary manifold. The system may further comprise a drape configured to be positioned over at least a portion of the apparatus and the primary manifold. The drape may be configured to seal to tissue adjacent to the tissue site to form a sealed space. The system may further comprise a negative-pressure source configured to provide negative pressure to the sealed space.

Further, in some example embodiments, a method of treating a tissue site with negative pressure may include positioning the apparatus including the primary manifold proximate to the tissue site; applying negative pressure to a sealed space at the tissue site including the apparatus and the primary manifold; and moving the primary manifold to the extended state by operation of the negative pressure, wherein the bottom surface of the primary manifold is configured to form a convex shape in conformity with the tissue site when the primary manifold is in the extended state.

Further, in some example embodiments, a method of treating a tissue site with negative pressure may include positioning the apparatus including the primary manifold proximate to the tissue site; applying negative pressure to a sealed space at the tissue site including the apparatus and the primary manifold; and extending one or more extension zones outward from the bottom surface of the primary manifold toward the tissue site.

In some example embodiments, a method of treating a tissue site according to this disclosure may include observing the tissue site through one or more openings disposed through the primary manifold. Alternatively or additionally, some example methods for treating a tissue site according to this disclosure may include observing the tissue site through a transparent material forming at least a portion of the primary manifold.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment and instillation treatment in accordance with this specification;

FIG. 2 is an exploded view of an example embodiment of a tissue interface, illustrating additional details that may be associated with some embodiments of the therapy system of FIG. 1;

FIG. 3 is an isometric view of an assembled example of the tissue interface of FIG. 2;

FIG. 4 is a cross-sectional view of the example tissue interface of FIG. 3 taken at line 4-4;

FIG. 5 is a bottom view illustrating details that may be associated with some embodiments of the example tissue interface of FIG. 2;

FIG. 6 is a bottom view illustrating details that may be associated with some embodiments of the example tissue interface of FIG. 2;

FIG. 7A is a top view of an example embodiment of a primary manifold in accordance with this specification;

FIG. 7B is an isometric partial view of some embodiments of the primary manifold of FIG. 7A;

FIG. 8A is a top view of an example embodiment of another primary manifold in accordance with this specification;

FIG. 8B is an isometric partial view of some embodiments of the primary manifold of FIG. 8A;

FIG. 9A is a bottom view of an example embodiment of another primary manifold in accordance with this specification;

FIG. 9B is an isometric partial view of some embodiments of the primary manifold of FIG. 9A;

FIG. 10A is a bottom view of an example embodiment of another primary manifold in accordance with this specification;

FIG. 10B is an isometric partial view of some embodiments of the primary manifold of FIG. 10A;

FIG. 11 is an exploded view of an example embodiment of a dressing including the tissue interface of FIG. 2, illustrating additional details that may be associated with some embodiments of the therapy system of FIG. 1;

FIG. 12 is an isometric view of an assembled example of the dressing of FIG. 11;

FIG. 13 is a cross-sectional view of the example dressing of FIG. 12, taken at line 13-13, applied to a tissue site, and illustrating additional details that may be associated with the therapy system of FIG. 1, in accordance with this specification;

FIG. 14A is a detail view, taken at reference FIG. 14A in FIG. 13, illustrating details that may be associated with some example embodiments of the example dressing of FIG. 13;

FIG. 14B illustrates additional details that may be associated with the detail view of FIG. 14A in some embodiments of the dressing of FIG. 13;

FIG. 15 is an isometric view of an assembled example of the tissue interface of FIG. 2 in accordance with this specification;

FIG. 16 is an isometric view of an example embodiment of the primary manifold in accordance with this specification;

FIG. 17 is an exploded view of an example of the dressing of FIG. 1, illustrating additional details that may be associated with some embodiments;

FIG. 17A is an isometric view of an assembled example of the dressing of FIG. 17;

FIG. 18 is a top view of the dressing of FIG. 17, as assembled, illustrating details that may be associated with some embodiments;

FIG. 19 is a bottom view of the dressing of FIG. 17, as assembled, illustrating details that may be associated with some embodiments;

FIG. 20 is a schematic view illustrating an example configuration of fluid passages that may be associated with some embodiments of dressings in accordance with this specification;

FIG. 21 is a schematic view of another example configuration of fluid passages;

FIG. 22 is a schematic view of another example configuration of fluid passages;

FIG. 23 is a schematic view of another example configuration of fluid passages;

FIG. 24 is a schematic view of another example configuration of fluid passages;

FIG. 25 is a schematic view of another example configuration of fluid passages;

FIG. 26 is a schematic view of another example configuration of fluid passages;

FIG. 27 is a schematic view of another example configuration of fluid passages;

FIG. 28 is a schematic view of another example configuration of fluid passages;

FIG. 29 is a schematic view of another example configuration of fluid passages;

FIG. 30 is a schematic view of another example configuration of fluid passages;

FIG. 31 is a schematic view of another example configuration of fluid passages;

FIG. 32 is a cross-sectional view of the example dressing of FIG. 17A, taken at line 32-32, applied to the example tissue site, and illustrating additional details associated with the therapy system of FIG. 1, in accordance with this specification;

FIG. 32A is a detail view, taken at reference FIG. 32A in FIG. 32, illustrating details that may be associated with some example embodiments of the example dressing of FIG. 32;

FIG. 32B illustrates additional details that may be associated with the detail view of FIG. 32A in some embodiments of the dressing of FIG. 32;

FIG. 33A is a top, plan view of another example embodiment of a primary manifold in accordance with this specification;

FIG. 33B is an isometric view of the example primary manifold of FIG. 33A;

FIG. 34A is a cross-section of the example primary manifold of FIG. 33A, taken at line 34A-34A as shown in FIG. 33A, in a retracted state;

FIG. 34B depicts the cross-section of the primary manifold of FIG. 34A, shown in an extended state;

FIG. 35A is a cross-section of an example pleat, taken at line 35A-35A in FIG. 33A, that may form an apparatus including the primary manifold of FIG. 33A and film layers, shown in a retracted state;

FIG. 35B is a cross-section of the apparatus of FIG. 35A including the example pleat of FIG. 33A and the film layers shown in an extended state;

FIG. 36A is a cross-section of another example pleat that may be associated with some embodiments of the primary manifold and an apparatus including the primary manifold and film layers, shown in a retracted state;

FIG. 36B is a cross-section of the example pleat of FIG. 36A shown in an extended state with the film layers; and

FIG. 37 is a cross-sectional view of an example dressing including the example primary manifold of FIG. 33A applied to a tissue site and illustrating additional details associated with the therapy system of FIG. 1, in accordance with this specification.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

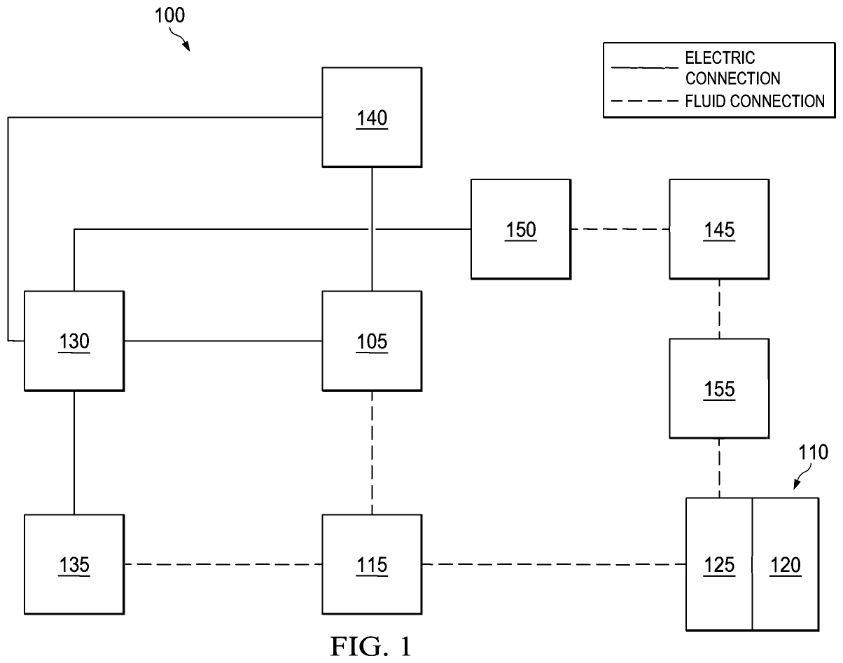

FIG. 1 is a block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface or connector may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

The therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source such as a positive-pressure source 150, a negative-pressure source such as the negative-pressure source 105, or both in some embodiments. A regulator, such as an instillation regulator 155, may also be fluidly coupled to the solution source 145 and the dressing 110 to ensure proper dosage of instillation solution (e.g. saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, the solution source 145, and other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” or “reduced pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure across the tissue interface 120, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may be substantially clear or optically transparent. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). In illustrative embodiments, the adhesive may be substantially clear or optically transparent. Thicker adhesives, or combinations of adhesives, may be applied in some embodiments to improve the seal and reduce leaks. Example embodiments of an attachment device may include a double-sided tape, paste, hydrocolloid, hydrogel, silicone gel, or organogel.

The solution source 145 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

In some embodiments, the controller 130 may have a continuous pressure mode, in which the negative-pressure source 105 is operated to provide a constant target negative pressure for the duration of treatment or until manually deactivated. Additionally or alternatively, the controller may have an intermittent pressure mode. For example, the controller 130 can operate the negative-pressure source 105 to cycle between a target pressure and atmospheric pressure. For example, the target pressure may be set at a value of 135 mmHg for a specified period of time (e.g., 5 min), followed by a specified period of time (e.g., 2 min) of deactivation. The cycle can be repeated by activating the negative-pressure source 105, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time may be a value substantially equal to the initial rise time.

In some example dynamic pressure control modes, the target pressure can vary with time. For example, the target pressure may vary in the form of a triangular waveform, varying between a negative pressure of 50 and 135 mmHg with a rise time set at a rate of +25 mmHg/min. and a descent time set at −25 mmHg/min. In other embodiments of the therapy system 100, the triangular waveform may vary between negative pressure of 25 and 135 mmHg with a rise time set at a rate of +30 mmHg/min and a descent time set at −30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

In some embodiments, the controller 130 may receive and process data, such as data related to instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second to 30 minutes. The controller 130 may also control the operation of one or more components of the therapy system 100 to instill solution. For example, the controller 130 may manage fluid distributed from the solution source 145 to the tissue interface 120. In some embodiments, fluid may be instilled to a tissue site by applying a negative pressure from the negative-pressure source 105 to reduce the pressure at the tissue site, drawing solution into the tissue interface 120. In some embodiments, solution may be instilled to a tissue site by applying a positive pressure from the positive-pressure source 150 to move solution from the solution source 145 to the tissue interface 120. Additionally or alternatively, the solution source 145 may be elevated to a height sufficient to allow gravity to move solution into the tissue interface 120.

The controller 130 may also control the fluid dynamics of instillation by providing a continuous flow of solution or an intermittent flow of solution. Negative pressure may be applied to provide either continuous flow or intermittent flow of solution. The application of negative pressure may be implemented to provide a continuous pressure mode of operation to achieve a continuous flow rate of instillation solution through the tissue interface 120, or it may be implemented to provide a dynamic pressure mode of operation to vary the flow rate of instillation solution through the tissue interface 120. In an intermittent mode, a specific fill volume and dwell time may be provided, depending, for example, on the type of tissue site being treated and the type of dressing being utilized. After or during instillation of solution, negative-pressure treatment may be applied. The controller 130 may be utilized to select a mode of operation and the duration of the negative pressure treatment before commencing another instillation cycle.

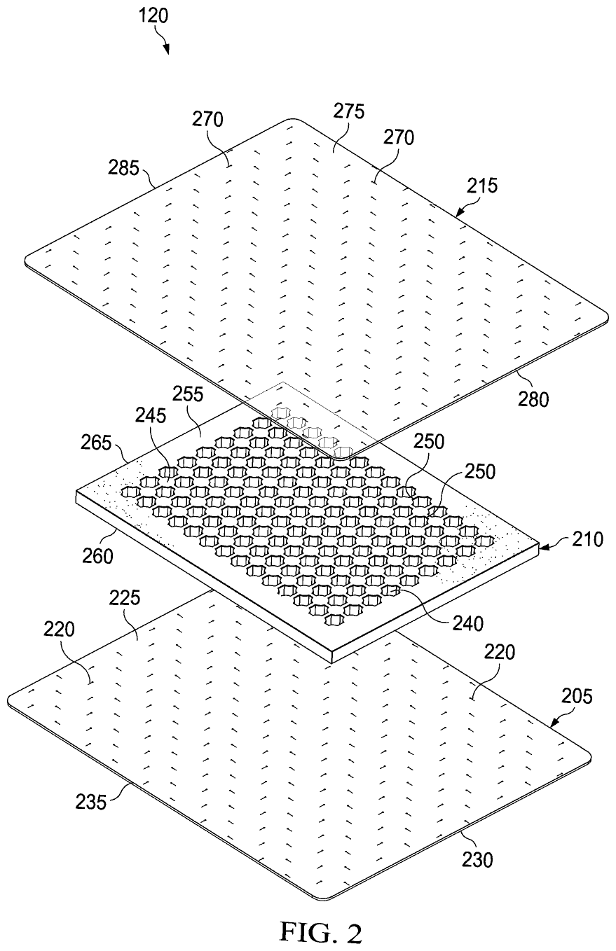

FIG. 2 is an exploded view of an example of the tissue interface 120 of FIG. 1, illustrating additional details that may be associated with some embodiments in which the tissue interface 120 comprises more than one layer. In the example of FIG. 2, the tissue interface 120 comprises a first polymer film or first film layer 205, a primary manifold 210, and a second polymer film or second film layer 215. In some embodiments, the first film layer 205 may be disposed adjacent to the primary manifold 210, and the second film layer 215 may be disposed adjacent to the primary manifold 210 opposite the first film layer 205. For example, the first film layer 205 and the primary manifold 210 may be stacked so that the first film layer 205 is in contact with the primary manifold 210. The second film layer 215 and the primary manifold 210 may be stacked so that the second film layer 215 is in contact with the primary manifold 210. In some embodiments, at least a portion of the first film layer 205 may be bonded to at least a portion of the second film layer 215. In illustrative embodiments, at least a portion of the primary manifold 210 may be bonded to at least a portion of at least one of the first film layer 205 and/or the second film layer 215.

The first film layer 205 may include a suitable structure for controlling or managing fluid flow. In some embodiments, the first film layer 205 may be a fluid-control layer which may include a liquid-impermeable, vapor permeable elastomeric material. In example embodiments, the first film layer 205 may include of a polymer film. For example, the first film layer 205 may include a polyolefin film, such as a polyethylene film. In illustrative embodiments, the first film layer 205 may be substantially clear or optically transparent. In some embodiments, the first film layer 205 may include the same material as the cover 125. In example embodiments, the first film layer 205 may include a biocompatible polyurethane film tested and certified according to the USP Class VI Standard. The first film layer 205 may also have a smooth or matte surface texture in some embodiments. A glossy or shiny finish better or equal to a grade B3 according to the SPI (Society of Plastics Industry) standards may be particularly advantageous for some applications. In some embodiments, variations in surface height may be limited to acceptable tolerances. For example, the surface of the first film layer 205 may be a substantially flat surface, with height variations limited to 0.2 millimeters over a centimeter.

In some embodiments, the first film layer 205 may be hydrophobic. The hydrophobicity of the first film layer 205 may vary, but may have a contact angle with water of at least ninety degrees in some embodiments. In some embodiments, the first film layer 205 may have a contact angle with water of no more than 150 degrees. For example, in some embodiments, the contact angle of the first film layer 205 may be in a range of at least 90 degrees to about 120 degrees, or in a range of at least 120 degrees to 150 degrees. Water contact angles may be measured used any standard apparatus. Although manual goniometers can be used to visually approximate contact angles, contact angle measuring instruments can often include an integrated system involving a level stage, a liquid dropper such as a syringe, a camera, and software designed to calculate contact angles more accurately and precisely, among other things. Non-limiting examples of such integrated systems may include the FTÅ125, FTÅ200, FTÅ2000, and FTÅ4000 systems, all commercially available from First Ten Angstroms, Inc., of Portsmouth, VA, and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles herein represent averages of 5-9 measured values, discarding both the highest and the lowest measured values. The hydrophobicity of the first film layer 205 may be further enhanced with a hydrophobic coating of other materials, such as silicones and fluorocarbons.

The first film layer 205 may also be suitable for welding to other layers, including the primary manifold 210 and the second film layer 215. For example, the first film layer 205 may be adapted for welding to polymers such as polyurethane, polyurethane films, and polyurethane foams using heat, radio-frequency (RF) welding, or other methods such as ultrasonic welding. RF welding may be particularly suitable for more polar materials, such as polyurethane, polyamides, polyesters, and acrylates. Sacrificial polar interfaces may be used to facilitate RF welding of less polar film materials, such as polyethylene.

The area density of the first film layer 205 may vary according to a prescribed therapy or application. In some embodiments, an area density of less than 40 grams per square meter may be suitable, and an area density of about 20-30 grams per square meter may be particularly advantageous for some applications.

In some embodiments, for example, the first film layer 205 may include a hydrophobic polymer, such as a polyethylene film. The simple and inert structure of polyethylene can provide a surface that interacts little, if any, with biological tissues and fluids, providing a surface that may encourage the free flow of liquids and low adherence, which can be particularly advantageous for many applications. Other suitable polymeric films include polyurethanes, acrylics, polyolefin (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate, styreneics, silicones, fluoropolymers, and acetates. A thickness between about 20 micrometers and about 500 micrometers may be suitable for many applications. For example, thicknesses of 23 micrometers, 25 micrometers, 100 micrometers, 250 micrometers, 300 micrometers, and 500 micrometers may be suitable for particular applications. More polar films suitable for laminating to a polyethylene film include polyamide, co-polyesters, ionomers, and acrylics. To aid in the bond between a polyethylene and polar film, tie layers may be used, such as ethylene vinyl acetate, or modified polyurethanes. An ethyl methyl acrylate (EMA) film may also have suitable hydrophobic and welding properties for some configurations.

As illustrated in the example of FIG. 2, the first film layer 205 may have one or more fluid passages 220, which can be distributed uniformly or randomly across the first film layer 205. The fluid passages 220 may be bi-directional and pressure-responsive. For example, each of the fluid passages 220 generally may be an elastic passage that is normally unstrained to substantially reduce liquid flow, and can expand or open in response to a pressure gradient. In some embodiments, the fluid passages 220 may include or may be perforations in the first film layer 205. The fluid passages 220 or perforations may be formed by removing material from the first film layer 205. For example, the fluid passages 220 or the perforations may be formed by cutting through the first film layer 205, which may also deform the edges of the perforations in some embodiments. In the absence of a pressure gradient across the perforations, the passages may be sufficiently small to form a seal or fluid restriction, which can substantially reduce or prevent liquid flow. Additionally or alternatively, one or more of the fluid passages 220 may include or may be a fenestration configured as an elastomeric valve that is normally closed when unstrained to substantially prevent liquid flow, and can open in response to a pressure gradient. Fenestrations may also be formed by removing material from the first film layer 205, but the amount of material removed and the resulting dimensions of the fenestrations may be up to an order of magnitude less than perforations, and may not deform the edges.

For example, some embodiments of the fluid passages 220 may include one or more slits, slots or combinations of slits and slots in the first film layer 205. In some examples, the fluid passages 220 may include linear slots having a length less than 5 millimeters and a width less than 2 millimeter. The length may be at least 2 millimeters, and the width may be at least 0.5 millimeters in some embodiments. A length in a range of about 2 millimeters to about 5 millimeters and a width in a range of about 0.5 millimeters to about 2 millimeters may be particularly suitable for many applications, and a tolerance of about 0.1 millimeters may also be acceptable. For example, a length of 3 mm may be suitable. Such dimensions and tolerances may be achieved with a laser cutter, for example. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow. In illustrative embodiments, the fluid passages 220 may comprise or consist of linear slits having a length of less than 5 millimeters. For example, the length may be at least 2 millimeters. A length in a range of about 2 millimeters to about 5 millimeters may be particularly suitable for many applications, and a tolerance of about 0.1 millimeters may also be acceptable. For example, a length of 3 mm may be suitable. In some embodiments, the first film layer 205 may comprise a top surface 225 opposite a bottom surface 230. The first film layer 205 may additionally comprise a periphery 235 at an outer perimeter of the first film layer 205. In illustrative embodiments, the fluid passages 220 may be circular, or any other suitable shape.

In some examples, the primary manifold 210 may be or may include a flexible grid structure. The flexible grid structure may be formed of or include a variety of materials, such as, without limitation a polymer, foam, or a combination of polymer and foam. Some examples of the primary manifold 210 may include a plurality of sections without polymer and/or foam material which form a plurality of manifold openings or windows 240. The manifold openings or windows 240 may be formed through the primary manifold 210, allowing the user to see through the primary manifold 210. In illustrative embodiments, the manifold openings or windows 240 may be defined as regions of the primary manifold 210 without material. For example, the manifold openings or windows 240 may also form flow channels, facilitating fluid communication and flow through a top surface 255 and a bottom surface 260 of the primary manifold 210. In illustrative embodiments, the primary manifold 210 comprises a molded or cast polymer, including but not limited to polyurethane or silicone based materials with a hardness in a range of about Shore 10A to about Shore 60A. For example, the primary manifold 210 may be formed from a polyurethane or silicone based material with a hardness in a range of Shore 20A to Shore 40A. A polymer with a hardness of Shore 10A may be suitable for particular applications. According to illustrative embodiments, the windows 240 may have a polygonal or circular frame. For example, the windows 240 may have a cross-shaped frame or a quatrefoil-shaped frame. In example embodiments, the frames for the windows 240 may be formed from regular shapes such as triangles, squares, pentagons, hexagons, or any other regular shape. In some embodiments, the frames for windows 240 may be formed from irregular shapes. According to example embodiments, the windows 240 may have a width in a range of about 8 millimeters to about 15 millimeters.

As illustrated in the example of FIG. 2, the primary manifold 210 may be formed from a single, substantially uniform material. The primary manifold 210 may comprise a plurality of primary nodes 245 arranged in a grid pattern. For example, the plurality of primary nodes 245 may be arranged in a pattern of rows and columns. Each primary node 245 within a row may be connected to at least one adjacent primary node 245 by a link 250. The centroid of each primary node 245 within a row may be aligned with a long axis of each link 250 connecting the primary nodes 245 within a row. In example embodiments, each primary node 245 within a column may be connected or linked to at least one adjacent primary node 245 by link 250. The centroid of each primary node 245 within a column may be aligned with a long axis of each link 250 connecting the primary nodes 245 within a row. In example embodiments, the links 250 within each row may be parallel with the links 250 within each other row. In example embodiments, the links 250 within each column may be parallel with the links 250 within each other column. For example, the links 250 within each column may be substantially orthogonal to the links 250 within each row. As illustrated by the example of FIG. 2, the top surfaces of the primary nodes 245 and the top surfaces of the links 250 may be substantially coplanar with the top surface 255 of the primary manifold 210. In some embodiments, the bottom surfaces of the primary nodes 245 and the bottom surfaces of the links 250 may be substantially coplanar with the bottom surface 260 of the primary manifold 210. In example embodiments, the plane formed by the top surface 255 of the primary manifold 210 may be substantially parallel with the plane formed by the bottom surface 260 of the primary manifold 210. The primary manifold 210 may additionally comprise a periphery 265 formed at an outer perimeter of primary manifold 210.

According to example embodiments, the primary nodes 245 may have a substantially circular profile in the plane formed by the top surface 255 of the primary manifold 210. For example, the circular profiles of the primary nodes 245 may have a diameter in a range of about 4 mm to about 12 mm. In example embodiments, the links 250 may have a substantially rectangular profile in the plane formed by the top surface 255 of the primary manifold 210. For example, the substantially rectangular profiles of the links 250 may have a length in a range of about 8 mm to about 15 mm.

In some embodiments, the primary nodes 245 may be arranged in a hexagonal or circular pattern, or any suitable pattern. In illustrative embodiments, the primary nodes may be any suitable three-dimensional shape. In some embodiments, the windows 240 may be framed by triangles, squares, rectangles, crosses, polygons, quatrefoils, or any other suitable shapes.

In example embodiments where the primary manifold 210 comprises a foam, a porous foam with an open-cell structure may be used. For example, a felted foam may be used. The porous foam or felted foam may have interconnected fluid pathways, for example, channels. Examples of suitable foams may include may include cellular foam, including open-cell foam such as reticulated foam; porous tissue collections; and other porous material such as gauze or felted mat that generally include pores, edges, and/or walls. In some embodiments, the primary manifold 210 may be formed by a felting process. Any porous foam suitable for felting may be used, including the example foams mentioned herein, such as GRANUFOAM™. Felting comprises a thermoforming process that permanently compresses a foam to increase the density of the foam while maintaining interconnected pathways. Felting may be performed by any known methods, which may include applying heat and pressure to a porous material or foam material. Some methods may include compressing a foam blank between one or more heated platens or dies (not shown) for a specified period of time and at a specified temperature. The direction of compression may be along the thickness of the foam blank. For example, the primary manifold 210 may be compressed in a direction substantially normal to the plane formed by the top surface 255 or the bottom surface 260 of the primary manifold 210.

The period of time of compression may range from 10 minutes up to 24 hours, though the time period may be more or less depending on the specific type of porous material used. Further, in some examples, the temperature may range between 120° C. to 260° C. Generally, the lower the temperature of the platen, the longer a porous material must be held in compression. After the specified time period has elapsed, the pressure and heat will form a felted structure or surface on or through the porous material or a portion of the porous material.

The felting process may alter certain properties of the original material, including pore shape and/or size, elasticity, density, and density distribution. For example, struts that define pores in the foam may be deformed during the felting process, resulting in flattened pore shapes. The deformed struts can also decrease the elasticity of the foam. The density of the foam is generally increased by felting. In some embodiments, contact with hot-press platens in the felting process can also result in a density gradient in which the density is greater at the surface and the pores size is smaller at the surface. In some embodiments, the felted structure may be comparatively smoother than any unfinished or non-felted surface or portion of the porous material. Further, the pores in the felted structure may be smaller than the pores throughout any unfinished or non-felted surface or portion of the porous material. In some examples, the felted structure may be applied to all surfaces or portions of the porous material. Further, in some examples, the felted structure may extend into or through an entire thickness of the porous material such that the all of the porous material is felted.

A felted foam may be characterized by a firmness factor, which is indicative of the compression of the foam. The firmness factor of a felted foam can be specified as the ratio of original thickness to final thickness. A compressed or felted foam may have a firmness factor greater than 1. The degree of compression may affect the physical properties of the felted foam. For example, felted foam has an increased effective density compared to a foam of the same material that is not felted. The felting process can also affect fluid-to-foam interactions. For example, as the density increases, compressibility or collapse may decrease. Therefore, foams which have different compressibility or collapse may have different firmness factors. In some example embodiments, a firmness factor can range from about 2 to about 10, preferably about 3 to about 5. For example, the firmness factor of the primary manifold 210 felted foam may be about 5 in some embodiments. There is a general linear relationship between firmness level, density, pore size (or pores per inch) and compressibility. For example, foam that is felted to a firmness factor of 3 will show a three-fold density increase and compress to about a third of its original thickness.

As illustrated by the example of FIG. 2, the second film layer 215 may comprise or consist essentially of a means for controlling or managing fluid flow. In some embodiments, the second film layer 215 may be a fluid-control layer comprising or consisting essentially of a liquid-impermeable, vapor permeable elastomeric material. In example embodiments, the second film layer 215 may comprise or consist essentially of a polymer film. For example, the second film layer 215 may comprise or consist essentially of a polyolefin film, such as a polyethylene film. In some embodiments, the second film layer 215 may comprise or consist essentially of the same material as the first film layer 205. The second film layer 215 may also have a smooth or matte surface texture in some embodiments. A glossy or shiny finish better or equal to a grade B3 according to the SPI (Society of Plastics Industry) standards may be particularly advantageous for some applications. In some embodiments, variations in surface height may be limited to acceptable tolerances. For example, the surface of the second film layer 215 may be a substantially flat surface, with height variations limited to 0.2 millimeters over a centimeter. In example embodiments, the second film layer 215 may be hydrophobic. For example, the second film layer 215 may have a contact angle with water of no more than 150 degrees. For example, the contact angle of the second film layer 215 may have a contact angle in a range of at least 90 degrees to about 120 degrees, or in a range of at least 120 degrees to 150 degrees.

The second film layer 215 may also be suitable for welding to other layers, including the first film layer 205 and the primary manifold 210. For example, the second film layer 215 may be adapted for welding to polymers such as polyurethane, polyurethane films, and polyurethane foams using heat, radio-frequency (RF) welding, or other methods such as ultrasonic welding. RF welding may be particularly suitable for more polar materials, such as polyurethane, polyamides, polyesters, and acrylates. Sacrificial polar interfaces may be used to facilitate RF welding of less polar film materials, such as polyethylene. The area density of the second film layer 215 may vary according to a prescribed therapy or application. In some embodiments, an area density of less than 40 grams per square meter may be suitable, and an area density of about 20-30 grams per square meter may be particularly advantageous for some applications. In some embodiments, for example, the second film layer 215 may comprise or consist essentially of a hydrophobic polymer, such as a polyethylene film. Other suitable polymers include the polymeric films described previously with respect to the first film layer 205. A thickness between about 20 micrometers and about 500 micrometers may be suitable for many applications. For example, a thickness of 23 micrometers may be suitable for particular applications. In illustrative embodiments, a thickness of 25 micrometers may be suitable for particular applications. In some embodiments, the thickness of the second film layer 215 may be less than the thickness of the first film layer 205. The second film layer 215 may be substantially clear, or optically transparent. As illustrated in the example of FIG. 2, the second film layer 215 may have one or more fluid passages 270. Fluid passages 270 may be substantially similar to or the same as the fluid passages 220 described previously with respect to the first film layer 205. In some embodiments, the second film layer 215 may comprise a top surface 275 opposite a bottom surface 280. The second film layer 215 may additionally comprise a periphery 285 at an outer perimeter of the second film layer 215.

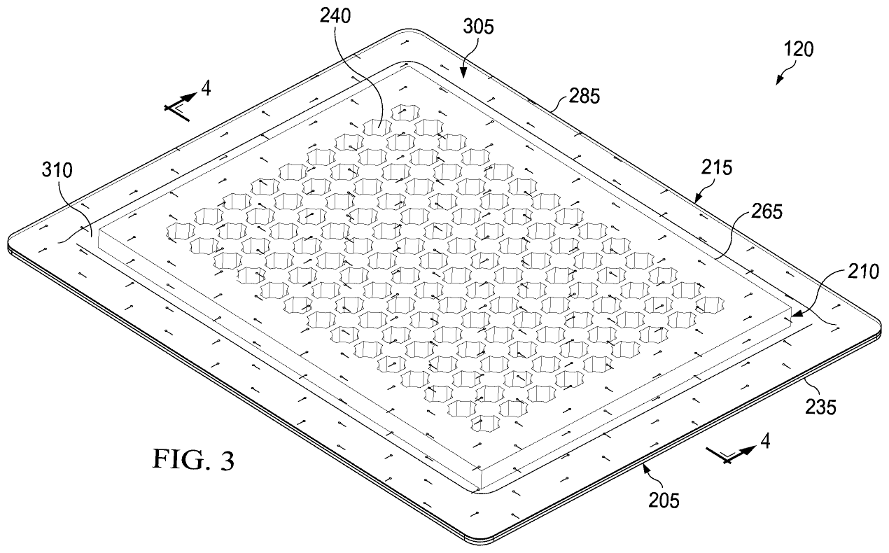

FIG. 3 shows an isometric view of some embodiments of the tissue interface 120 with the first film layer 205, primary manifold 210, and the second film layer 215 in assembled form. In illustrative embodiments, the periphery 235 of the first film layer 205 may be substantially coextensive with the periphery 285 of the second film layer 215. In some embodiments, a portion of the first film layer 205 near the periphery 235 of the first film layer 205 may be coupled, bonded, welded, or adhered to a portion of the second film layer 215 near the periphery 285 of the second film layer 215 at a border region 305 to define an interior space 310 of the tissue interface 120. Coupling may also include, mechanical, thermal, or chemical coupling (such as a chemical bond) in some contexts. The primary manifold 210 may be positioned within the interior space of the tissue interface 120.

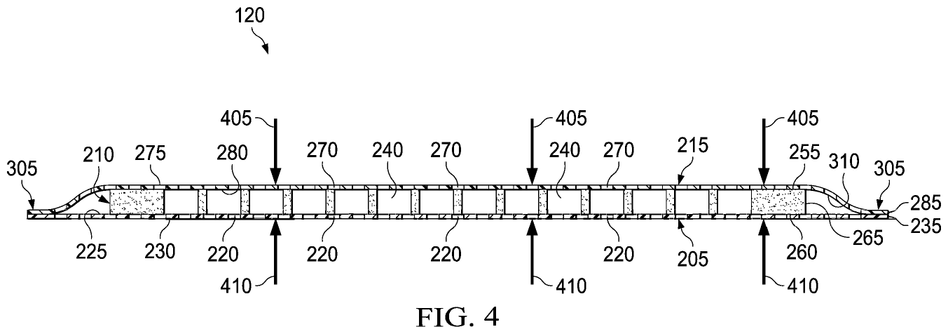

FIG. 4 shows a cross-sectional view of the example tissue interface 120 of FIG. 3 taken at line 4-4. Assembled, the top surface 225 of the first film layer 205 may be adjacent to the bottom surface 260 of the primary manifold 210. The top surface 255 of the primary manifold 210 may be adjacent to the bottom surface 280 of the second film layer 215. The portion of the first film layer 205 coupled, bonded, welded, or adhered to the portion of the second film layer 215 may form the border region 305 of the tissue interface 120. In illustrative embodiments, the primary manifold 210 may be positioned within the interior space 310 of the tissue interface 120. For example, the periphery 265 of the primary manifold 210 may be contained within the border region 305 and within the interior space 310. For example, the primary manifold 210 may be contained by the top surface 225 of the first film layer 205, the bottom surface 280 of the second film layer 215, and the border region 305. In assembled form, the user is able to see through the tissue interface 120 in a direction approximately normal to the plane formed by the top surface 275 of the second film layer 215 or the bottom surface 230 of the first film layer 205. For example, the user may see through the substantially clear or optically transparent first film layer 205, into and through the windows 240 of the primary manifold 210, and through the substantially clear or optically transparent second film layer 215.

In illustrative embodiments, fluid may be transported through the fluid passages 220 of the first film layer 205 and into the windows 240 of the primary manifold 210, and from the windows 240 through the fluid passages 270 of the second film layer 215. In example embodiments, fluid may be transported through the fluid passages 270 of the second film layer 215 and into the windows 240 of the primary manifold 210, and from the windows 240 through the fluid passages 220 of the first film layer 205. In some embodiments, fluid may be transported through the tissue interface 120. In example embodiments where the primary manifold 210 includes a porous material, fluid may be transported through the flow channels formed within the porous material of the primary manifold 210. In illustrative embodiments, the primary manifold 210 may be sufficiently stiff to resist substantial deformation when a first force 405 and a second force 410 is applied to the manifold. For example, the first force 405 may be substantially normal to the top surface 255 of the primary manifold 210, and the second force 410 may be substantially normal to the bottom surface 260 of the primary manifold 210. In example embodiments, the first force 405 and the second force 410 may be substantially opposite vectors. By preventing the primary manifold 210 from deforming in response to the first force 405 and/or the second force 410, the primary manifold 210 may keep the windows 240 substantially open in response to the applied forces 405 and/or 410.

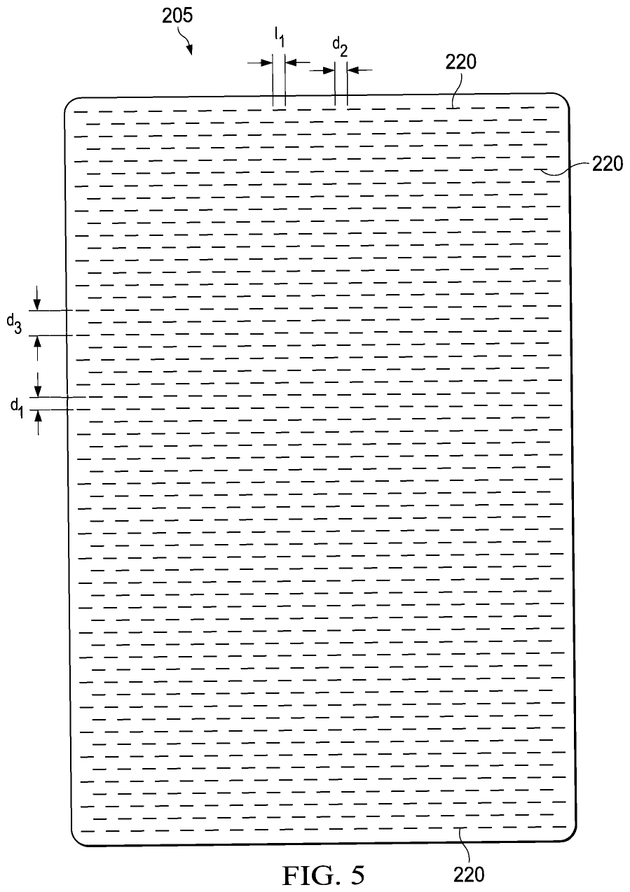

FIG. 5 is a bottom view illustrating details that may be associated with some embodiments of the example tissue interface 120 of FIG. 2. For example, FIG. 5 illustrates additional details that may be associated with some embodiments of the first film layer 205. As illustrated in the example of FIG. 5, the fluid passages 220 may each consist essentially of one or more slits having a length l₁. A length of about 3 millimeters may be particularly suitable for some embodiments. FIG. 5 additionally illustrates an example of a uniform distribution pattern of the fluid passages 220. In FIG. 5, the fluid passages 220 are substantially coextensive with the first film layer 205, and are distributed across the first film layer 205 in a grid of parallel rows and columns, in which the slits are also mutually parallel to each other. In some embodiments, the rows may be spaced a distance d₁. A distance of about 3 millimeters on center may be suitable for some embodiments. The fluid passages 220 within each of the rows may be spaced a distance d₂, which may be about 3 millimeters on center in some examples. The fluid passages 220 in adjacent rows may be aligned or offset in some embodiments. For example, adjacent rows may be offset, as illustrated in FIG. 5, so that the fluid passages 220 are aligned in alternating rows and separated by a distance d₃, which may be about 6 millimeters in some embodiments. The spacing of the fluid passages 220 may vary in some embodiments to increase the density of the fluid passages 220 according to therapeutic requirements. In some embodiments, a plurality of fluid passages 220 may align with the windows 240 of the primary manifold 210 when the tissue interface 120 is assembled. For example, a majority of fluid passages 220 may be aligned with the windows 240 of the primary manifold 210 to facilitate improved moisture transfer through the tissue interface 120 and to facilitate improved manifolding through the tissue interface 120. In illustrative embodiments, a majority of the fluid passages 220 may be aligned with the plurality of primary nodes 245 to improve manifolding around the primary nodes 245 by fluid passages 220 which route over the arc of the primary nodes 245.

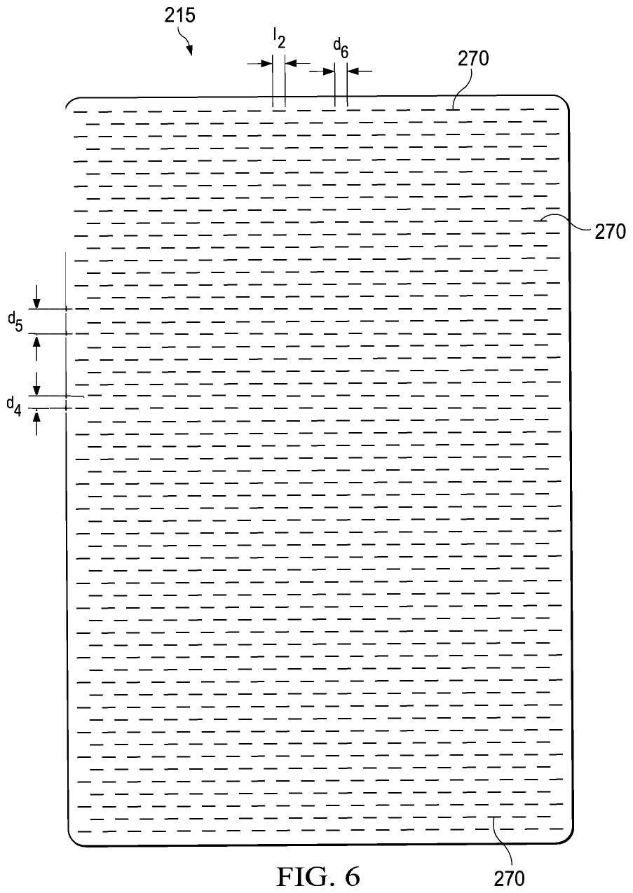

FIG. 6 is a bottom view illustrating details that may be associated with some embodiments of the example tissue interface 120 of FIG. 2. For example, FIG. 6 illustrates additional details that may be associated with some embodiments of the second film layer 215. As illustrated in the example of FIG. 6, the fluid passages 270 may each consist essentially of one or more slits having a length l₂. A length of about 3 millimeters may be particularly suitable for some embodiments. FIG. 6 additionally illustrates an example of a uniform distribution pattern of the fluid passages 270. In FIG. 6, the fluid passages 270 are substantially coextensive with the second film layer 215, and are distributed across the second film layer 215 in a grid of parallel rows and columns, in which the slits are also mutually parallel to each other. In some embodiments, the rows may be spaced a distance d₄. A distance of about 3 millimeters on center may be suitable for some embodiments. The fluid passages 270 within each of the rows may be spaced a distance (15, which may be about 3 millimeters on center in some examples. The fluid passages 270 in adjacent rows may be aligned or offset in some embodiments. For example, adjacent rows may be offset, as illustrated in FIG. 6, so that the fluid passages 270 are aligned in alternating rows and separated by a distance d₆, which may be about 6 millimeters in some embodiments. The spacing of the fluid passages 270 may vary in some embodiments to increase the density of the fluid passages 270 according to therapeutic requirements. In some embodiments, a plurality of fluid passages 270 may align with the windows 240 of the primary manifold 210 when the tissue interface 120 is assembled. For example, a majority of fluid passages 270 may be aligned with the windows 240 of the primary manifold 210 to facilitate improved moisture transfer through the tissue interface 120 and to facilitate improved manifolding through the tissue interface 120.

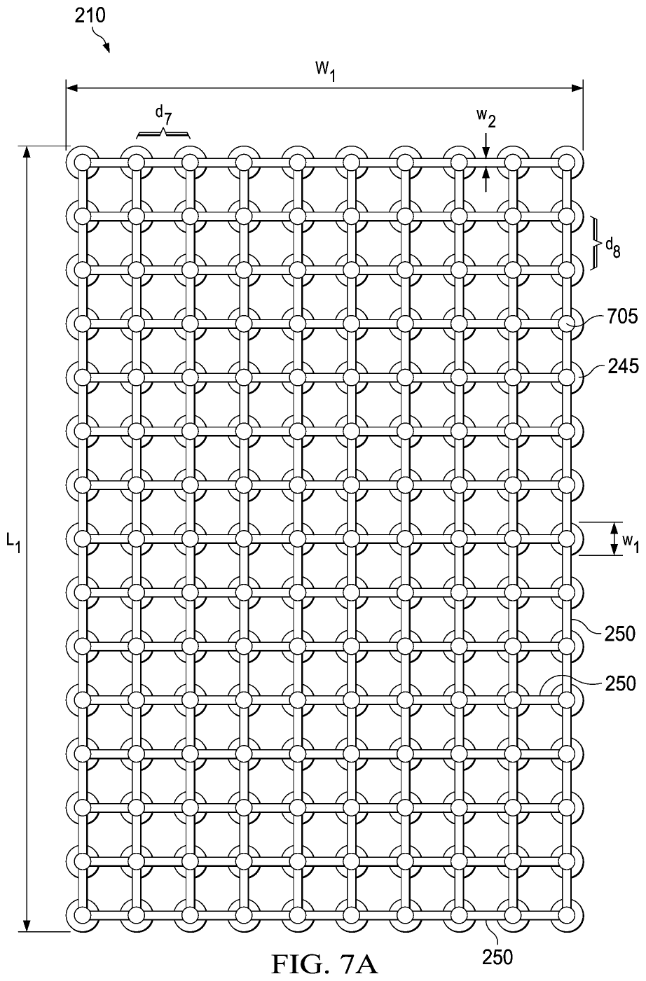

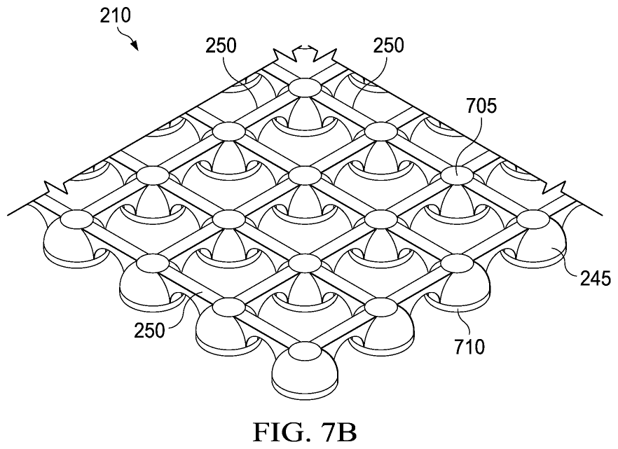

FIG. 7A is a top view of an example embodiment of a primary manifold 210, illustrating additional details that may be associated with some embodiments of the therapy system of FIG. 1. For example, the primary manifold 210 may comprise a plurality of primary nodes 245 arranged in a grid pattern. In some embodiments, the primary nodes 245 may be interconnected by a network of links 250. For example, each primary node 245 may be connected to at least one other primary node 245 by a link 250. In example embodiments, each link 250 may be substantially parallel with or substantially orthogonal to each other link 250 in a plane. For example, each link 250 connected to any one primary node 245 may be orthogonal to an adjacent link 250 connected to the same primary node 245. In illustrative embodiments, the primary nodes 245 are substantially hemispherical, and the primary manifold 210 may also include a cap portion 705 at the pole of the primary node 245. In example embodiments, each primary node 245 may be spaced a distance d₇ on-center in a first direction from an adjacent primary node 245. Each primary node 245 may be spaced a distance d₈ on-center from an adjacent primary node 245 in a second direction. In illustrative embodiments, the first direction may be orthogonal to the second direction in the same plane. In some embodiments, each primary node 245 may have a diameter w₁. In example embodiments, each link 250 may have a width w₁. In some embodiments, the primary manifold 210 may have an overall length L₁ and an overall width W₁. For example, according to some embodiments, d₇ may be about 13 mm, d₈ may be about 13 mm, w₁ may be about 8 mm, w₂ may be about 2 mm, L₁ may be about 182 mm, and W₁ may be about 117 mm. According to illustrative embodiments, the primary manifold 210 may include a plurality of windows 240 defined by the negative spaces or portions where there is not material when the primary manifold 210 is viewed from the top.

FIG. 7B is an isometric partial view of the primary manifold 210 of FIG. 7A. In example embodiments, each hemispherical primary node 245 may include a filleted or radiused portion 710 around the base of the primary node 245. In some embodiments, each component of the primary manifold 210, such as each primary node 245, each link 250, and each cap portion 705 may include the same material. For example, the primary manifold 210 may be formed from a molded or cast polymer material, such as a polyurethane or silicone based material having a hardness between about Shore 20A and about Shore 40A. For example, a silicone material with a hardness of about Shore 10A may be suitable for particular applications. In example embodiments, the primary manifold 210 may have an overall height H₁. For example, H₁ may be about 4 mm.

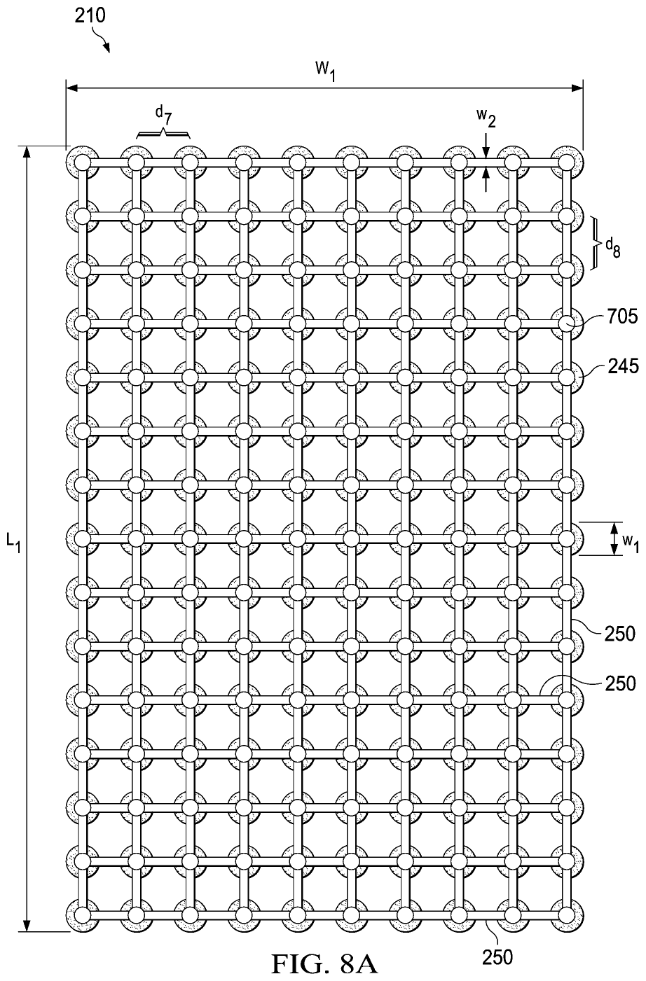

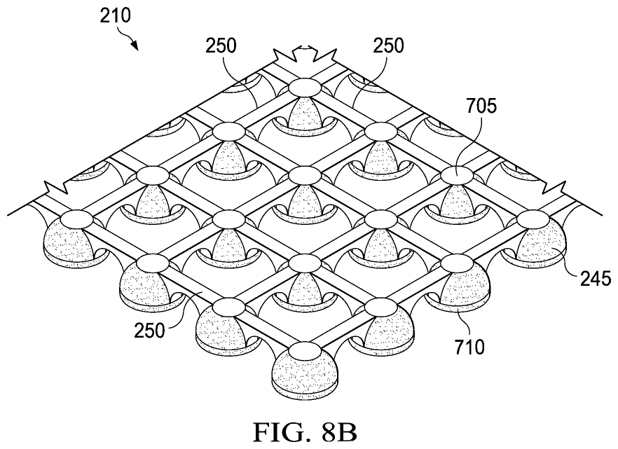

FIG. 8A is a top view of an example embodiment of a primary manifold 210, illustrating additional details that may be associated with some embodiments of the therapy system of FIG. 1. FIG. 8B is an isometric partial view of the primary manifold 210 of FIG. 8A. In example embodiments, each primary node 245 may be formed from a polymer having a lower Shore hardness level than each link 250 and each cap portion 705. For example, each primary node 245 may be formed from a silicone with a hardness of about Shore 10A, and each link 250 and each end cap portion 705 may be formed from a silicone with a hardness in a range of about Shore 20A to about Shore 40A.

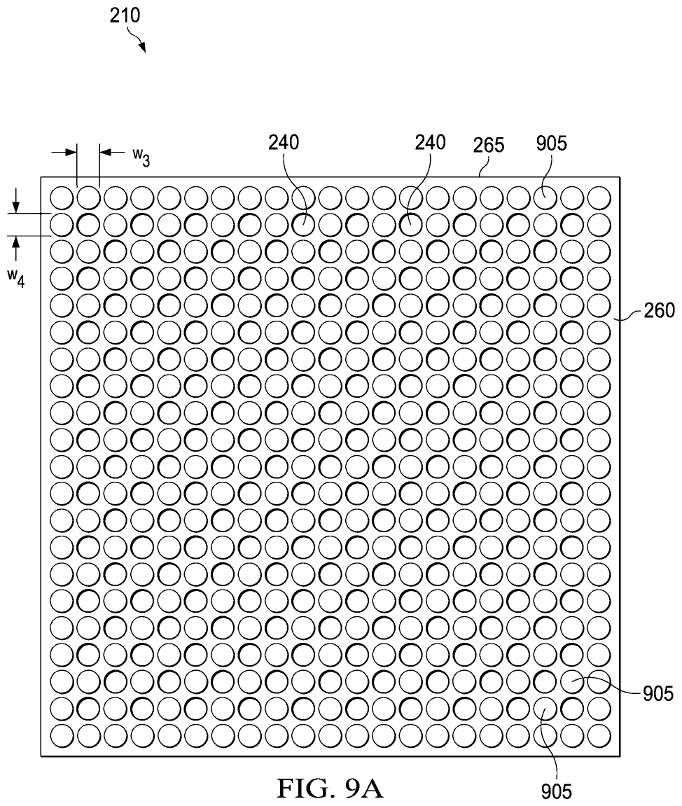

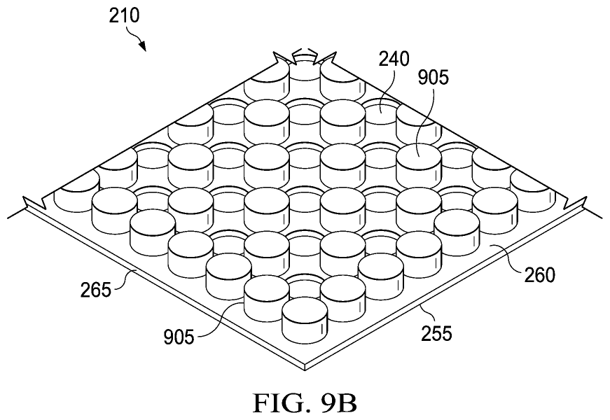

FIG. 9A is a bottom view of an example embodiment of the primary manifold 210, illustrating additional details that may be associated with the therapy system of FIG. 1. For example, the primary manifold 210 may be formed as a substantially sheet-like structure comprising a top surface 255 (not shown) and a bottom surface 260. For example, the primary manifold 210 may be formed from a sheet of polyurethane, such as a vacuum-formed sheet of polyurethane with a thickness of about 0.5 mm. In illustrative embodiments, the primary manifold 210 may be formed from a polymer material that is substantially clear or optically transparent, allowing the user to see through the primary manifold 210. Portions of the primary manifold 210 may be removed to form windows 240 in the primary manifold 210 in a grid pattern. A plurality of standoffs 905 may be formed on the primary manifold 210. The plurality of standoffs 905 may be formed around the periphery 265 of the primary manifold 210, and between each of the windows 240. For example, the plurality of standoffs 905 and the plurality of windows 240 may be arranged in a grid pattern. In illustrative embodiments, the standoffs 905 and windows 240 may be arranged in a pattern of rows and columns. For example, the center of each standoff 905 may be aligned with the center of each window 240 within a row. For example, the center of each standoff 905 within a column may be aligned with the center of each window 240 within the same row. In example embodiments, the rows and columns of nearest to the periphery 265 may comprise essentially of standoffs 905. In illustrative embodiments, inboard of the rows and columns of standoffs 905 nearest to the periphery 265, the pattern may alternate between standoffs 905 and windows 240 within each row. In example embodiments, inboard of the rows and columns of standoffs 905 nearest to the periphery 265, the pattern may alternate between standoffs 905 and windows 240 within each column. In some embodiments, the pattern may be arbitrarily chosen or random. In example embodiments, each standoff 905 may be substantially circular in profile in the plane of the bottom surface 260 of the primary manifold 210, and have a diameter w₃. In illustrative embodiments, each window 240 may be substantially circular in profile in the plane of the bottom surface 260 of the primary manifold 210, and have a diameter w₄. In example embodiments, w₃ may be substantially equal to w₄. In some embodiments, windows 240 may be square, or any suitable shape.

FIG. 9B is an isometric partial view of the primary manifold 210 of FIG. 9A. In some embodiments, the plurality of standoffs 905 comprises right cylinders which are formed on and protrude substantially away from the bottom surface 260 of the primary manifold 210 in a direction substantially normal to the bottom surface 260. In illustrative embodiments, the standoffs 905 may comprise any suitable shape.

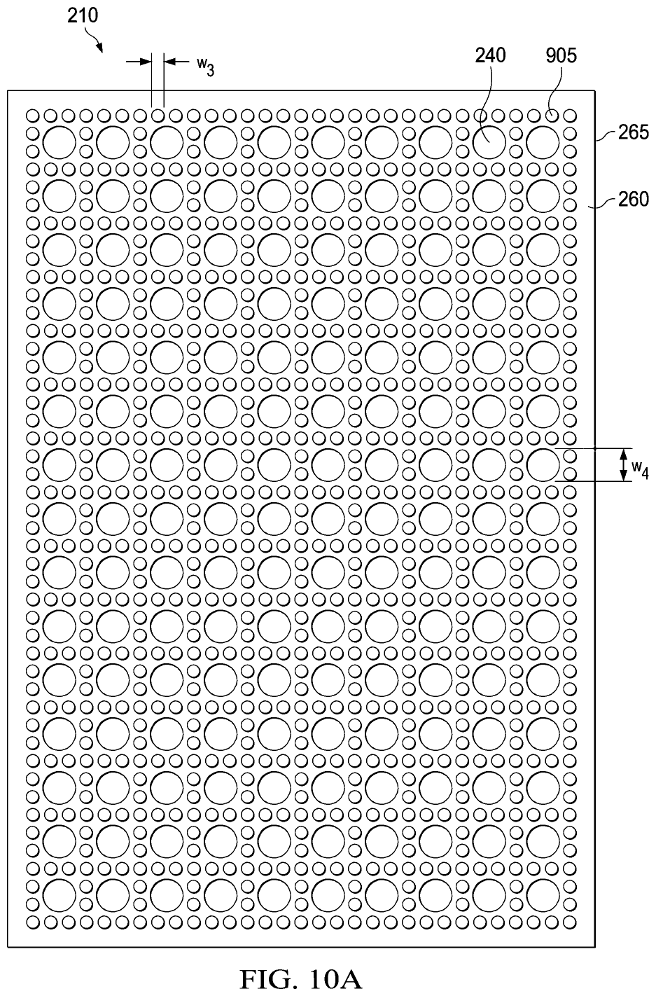

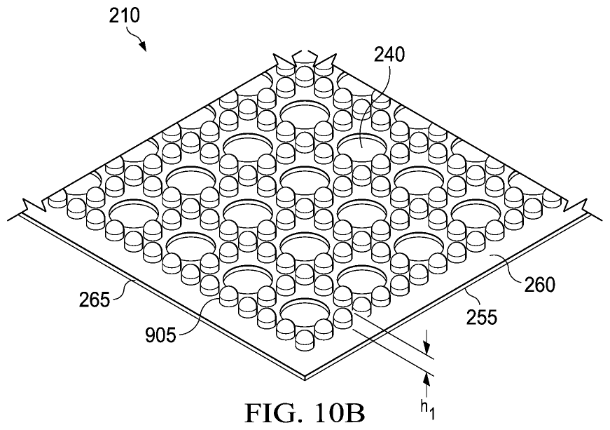

FIG. 10A is a bottom view of an example embodiment of the primary manifold 210, illustrating additional details that may be associated with the therapy system of FIG. 1. For example, the primary manifold 210 may be formed as a substantially sheet-like structure comprising a top surface 255 and a bottom surface 260 (not shown). For example, the primary manifold 210 may be formed from a sheet of polyurethane, such as a vacuum-formed sheet of polyurethane with a thickness of about 0.5 mm. In example embodiments, the primary manifold 210 may be formed from a polymer material that is substantially clear or optically transparent, allowing the user to see through the primary manifold 210. Windows 240 may be removed from the primary manifold and form a grid pattern. For example, the plurality of windows 240 may be arranged in a pattern of rows and columns. The center of each window 240 may be aligned with the center of each other window 240 within a row. The center of each window 240 may also be aligned with the center of each other window 240 within a column. In illustrative embodiments, a plurality of standoffs 905 may be formed on the primary manifold 210. The plurality of standoffs 905 may form a grid pattern. For example, the plurality of standoffs 905 may be arranged in a pattern of rows and columns. The center of each standoff 905 within a row may be aligned with the center of each other standoff 905 within the row. The center of each standoff 905 within a column may be aligned with the center of each other standoff 905 within a column. In some embodiments, each row of the plurality of windows 240 may be disposed in between two adjacent rows of the plurality of standoffs 905. In example embodiments, each column of the plurality of windows 240 may be disposed in between two adjacent columns of the plurality of standoffs 905. In some embodiments, the pattern of the plurality of windows 240 may be arbitrarily chosen or random. In illustrative embodiments, the pattern of the plurality of standoffs 905 may be arbitrarily chosen or random. In example embodiments, each standoff 905 may be substantially circular in profile in the plane of the bottom surface 260 of the primary manifold 210, and have a diameter w₃. In illustrative embodiments, each window 240 may be substantially circular in profile in the plane of the top surface 255 of the primary manifold 210, and have a diameter w₄. In example embodiments, w₃ may be substantially less than w₄. For example, w₃ may be about 3 mm in particular embodiments, and w₄ may be about 8 mm. In example embodiments, each standoff 905 within a row may be spaced a distance of about 4 mm on center from an adjacent standoff 905 within the row. In illustrative embodiments, each standoff 905 within a column may be spaced a distance of about 4 mm on center from an adjacent standoff 905 within the column. In some embodiments, windows 240 may be square, or any suitable shape.

FIG. 10B is an isometric view of the primary manifold 210 of FIG. 10A. In some embodiments, the plurality of standoffs 905 comprises right cylinders with hemispherical ends, such as half-capsules, which may be formed on and protrude substantially away from the bottom surface 260 of the primary manifold in a direction substantially normal to the bottom surface 260. In example embodiments, each of the plurality of standoffs 905 may have a height h₁. For example, in some embodiments, the height h₁ in a range of about 2.5 mm to about 3 mm may be suitable for particular applications. In illustrative embodiments, the standoffs 905 may comprise any suitable shape.

FIG. 11 is an exploded view of an example embodiment of a dressing 110 including the tissue interface 120 of FIG. 2, illustrating additional details that may be associated with some embodiments of the therapy system 100 of FIG. 1. In example embodiments, the dressing 110 may include the cover 125 and a secondary manifold 1105. In illustrative embodiments, the cover 125 may be substantially clear or optically transparent. In some embodiments, the secondary manifold 1105 generally comprises or consists essentially of a manifold or a manifold layer, which provides a means for collecting or distributing fluid across the dressing 110 under pressure. In some illustrative embodiments, the pathways of the secondary manifold 1105 may be interconnected to improve distribution or collection of fluids. In some illustrative embodiments, the secondary manifold 1105 may comprise or consist essentially of a porous material having interconnected fluid pathways. Examples of suitable porous material that comprise or can be adapted to form fluid pathways (e.g., channels) may include cellular foam, including open-cell foam such as reticulated foam, porous tissue collections, and other porous materials such as gauze or felted mat that generally includes pores, edges, and/or walls. Liquids, gels, and other foams may also include or be cured to include apertures and fluid pathways. In some embodiments, the secondary manifold 1105 may additionally or alternatively comprise projections that form interconnected fluid pathways. For example, the secondary manifold 1105 may be molded to provide surface projections that define interconnected fluid pathways.

In some embodiments, the secondary manifold 1105 may comprise or consist essentially of a reticulated foam having pore sizes and free volume that may vary according to needs of a prescribed therapy. For example, a reticulated foam having a free volume of at least 90% may be suitable for many therapy applications, and a foam having an average pore size in a range of 400-600 microns may be particularly suitable for some types of therapy. The tensile strength of the secondary manifold 1105 may also vary according to needs of a prescribed therapy. For example, the tensile strength of a foam may be increased for instillation of topical treatment solutions. The 25% compression load deflection of the secondary manifold 1105 may be at least 0.35 pounds per square inch, and the 65% compression load deflection may be at least 0.43 pounds per square inch. In some embodiments, the tensile strength of the secondary manifold 1105 may be at least 10 pounds per square inch. The secondary manifold 1105 may have a tear strength of at least 2.5 pounds per inch. In some embodiments, the secondary manifold 1105 may be a foam comprised of polyols such as polyester or polyether, isocyanate such as toluene diisocyanate, and polymerization modifiers such as amines and tin compounds. In some examples, the secondary manifold 1105 may be a reticulated polyurethane foam such as used in GRANUFOAM™ dressing or V.A.C. VERAFLO™ dressing, both available from KCl of San Antonio, Texas.

Other suitable materials for the secondary manifold 1105 may include non-woven fabrics (Libeltex, Freudenberg), three-dimensional (3D) polymeric structures (molded polymers, embossed and formed films, and fusion bonded films [Supracor]), and mesh, for example.

In some examples, the secondary manifold 1105 may include a 3D textile, such as various textiles commercially available from Baltex, Muller, and Heathcoates. A 3D textile of polyester fibers may be particularly advantageous for some embodiments. For example, the secondary manifold 1105 may comprise or consist essentially of a three-dimensional weave of polyester fibers. In some embodiments, the fibers may be elastic in at least two dimensions. A puncture-resistant fabric of polyester and cotton fibers having a weight of about 650 grams per square meter and a thickness of about 1-2 millimeters may be particularly advantageous for some embodiments. Such a puncture-resistant fabric may have a warp tensile strength of about 330-340 kilograms and a weft tensile strength of about 270-280 kilograms in some embodiments. Another particularly suitable material may be a polyester spacer fabric having a weight of about 470 grams per square meter, which may have a thickness of about 4-5 millimeters in some embodiments. Such a spacer fabric may have a compression strength of about 20-25 kilopascals (at 40% compression). Additionally or alternatively, the secondary manifold 1105 may comprise or consist of a material having substantial linear stretch properties, such as a polyester spacer fabric having 2-way stretch and a weight of about 380 grams per square meter. A suitable spacer fabric may have a thickness of about 3-4 millimeters, and may have a warp and weft tensile strength of about 30-40 kilograms in some embodiments. The fabric may have a close-woven layer of polyester on one or more opposing faces in some examples.

The secondary manifold 1105 generally has a first planar surface, such as a top surface 1110 opposite a second planar surface, such as a bottom surface 1115. The thickness of the secondary manifold 1105 between the top surface 1110 and the bottom surface 1115 may also vary according to the needs of a prescribed therapy. For example, the thickness of the secondary manifold 1105 may be decreased to relieve stress on other layers. The secondary manifold 1105 also comprises a periphery 1120 around an outer perimeter of the secondary manifold 1105. In some embodiments, a suitable foam secondary manifold 1105 may have a thickness in a range of about 5 millimeters to about 10 millimeters. In example embodiments, a fabric secondary manifold 1105, including 3D textiles and spacer fabrics, may have a thickness in a range of about 2 millimeters to about 8 millimeters.

The cover 125 generally has a first planar surface, such as a top surface 1125 opposite a bottom surface 1130. In example embodiments, at least a portion of the bottom surface 1130 of the cover 125 may be coated with an adhesive, such as an acrylic adhesive. The cover 125 may also comprise a periphery 1135 around an outer perimeter of the cover 125. An aperture 1140 may be formed in the cover 125. In some embodiments, the periphery 1135 of the cover 125 may be greater than the periphery 1120 of the secondary manifold 1105, the periphery 285 of the second film layer 215, the periphery 265 of the primary manifold 210, and the periphery 235 of the first film layer 205. For example, the periphery 1120 of the secondary manifold 1105, the periphery 285 of the second film layer 215, the periphery 265 of the primary manifold 210, and the periphery 235 of the first film layer 205 may be contained within the periphery 1135 of the cover 125. In example embodiments, the periphery 1120 of the secondary manifold 1105 may be contained within the periphery 1135 of the cover 125 and the periphery 285 of the second film layer 215.

FIG. 11 also illustrates one example of a fluid conductor 1145 and a dressing interface 1150. As shown in the example of FIG. 11, the fluid conductor 1145 may be a flexible tube, which can be fluidly coupled on one end to the dressing interface 1150. The dressing interface 1150 may be an elbow connector, as shown in the example of FIG. 11, which can be placed over the aperture 1140 in the cover 125 to provide a fluid path between the fluid conductor 1145 and the secondary manifold 1105.

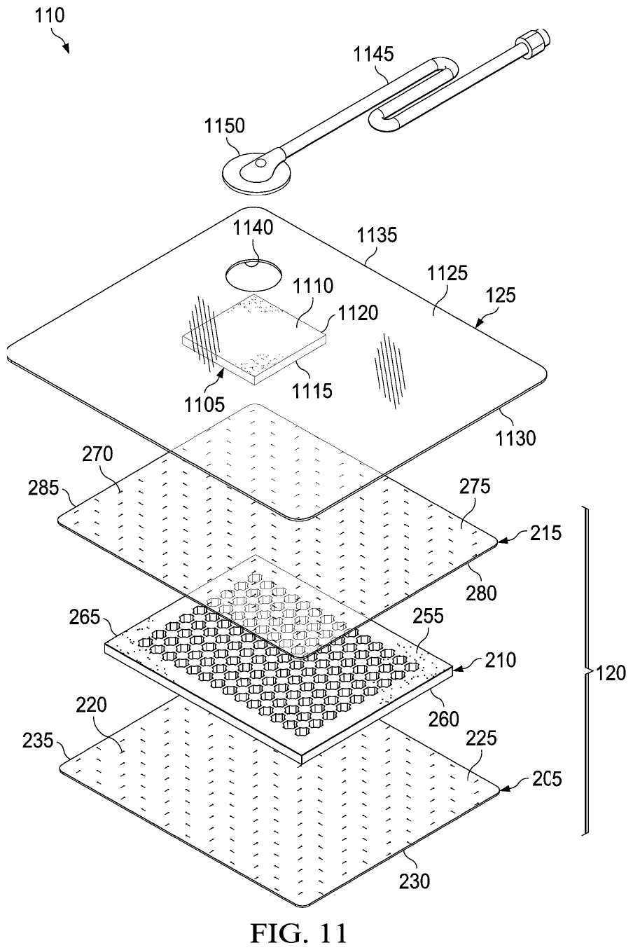

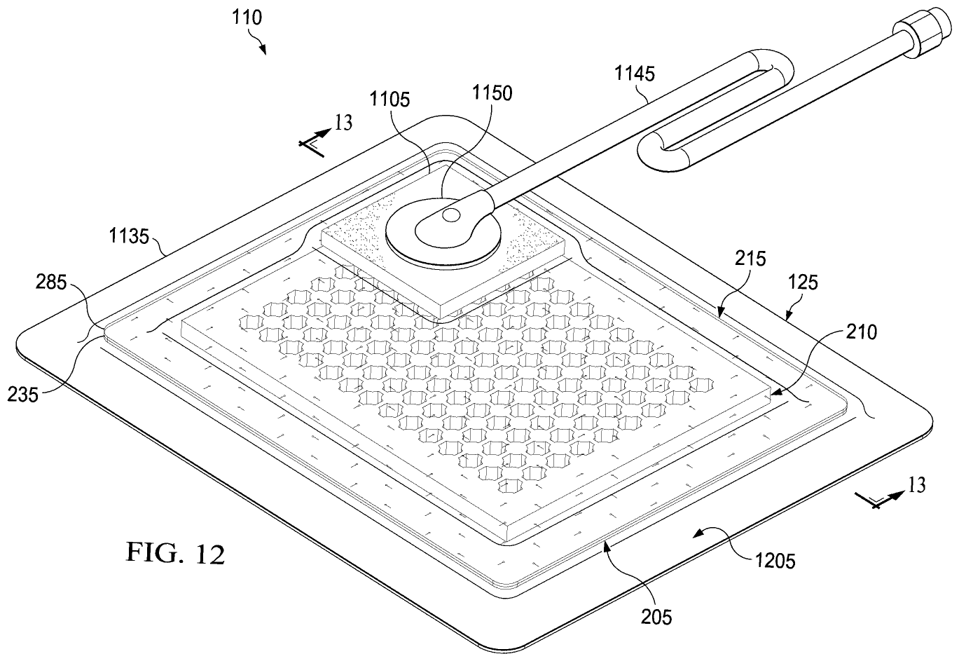

FIG. 12 is an isometric view of an assembled example of the dressing 110 of FIG. 11. As shown in the example of FIG. 12, the cover 125 may be substantially clear or optically transparent, allowing for visualization of the layers of the dressing 110 and through the dressing 110. In example embodiments, the periphery 1135 of the cover 125 extends past the periphery 235 of the first film layer 205 and the periphery 285 of the second film layer 215, defining a border region 1205 of the cover 125.

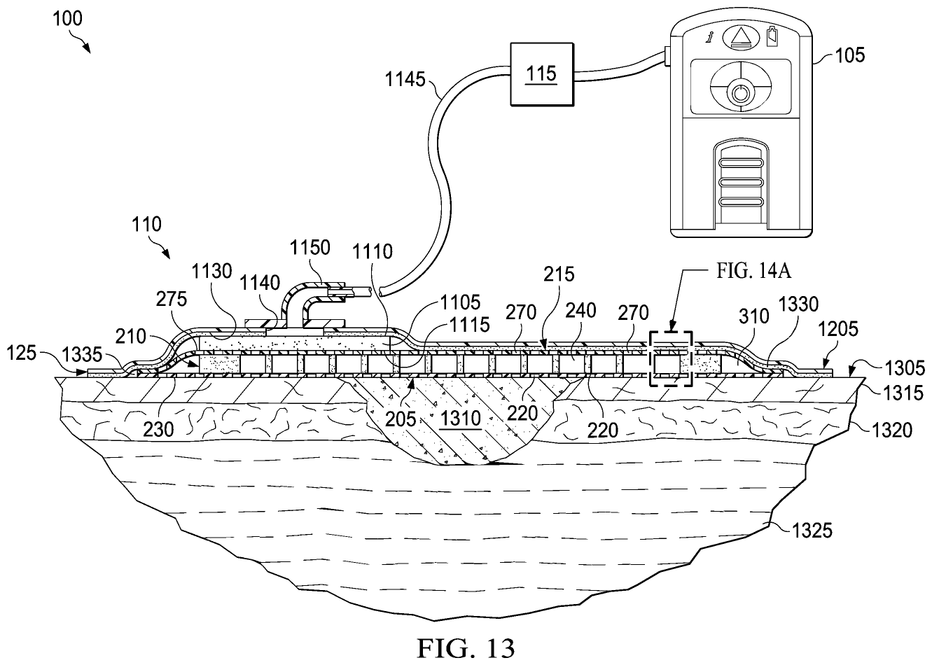

FIG. 13 is a cross-sectional view of the example dressing 110 of FIG. 12, taken at line 13-13, applied to an example tissue site, and illustrating additional details associated with the therapy system 100 of FIG. 1. In some embodiments, the dressing 110 may be configured to interface with a tissue site 1305. For example, the dressing 110 may be generally configured to be positioned adjacent to the tissue site 1305 and/or in contact with a portion of the tissue site 1305, substantially all of the tissue site 1305, or the tissue site 1305 in its entirety, or tissue around the tissue site 1305. In some examples, the tissue site 1305 may be or may include a defect or targeted treatment site, such as a wound, that may be partially or completely filled or covered by the dressing 110. In various embodiments, the dressing 110 may take many forms, and may have many sizes, shapes, or thicknesses depending on a variety of factors, such as the type of treatment being implemented or the nature and size of the tissue site 1305. For example, the size and the shape of the dressing 110 may be adapted to the contours of deep and irregularly shaped tissue sites and/or may be configured to be adapted to a given shape or contour. Moreover, in some embodiments, any or all of the surfaces of the dressing 110 may comprise projections, or an uneven, coarse, or jagged profile that can, for example, induce strains and stresses on the tissue site 1305, which may be effective to promote granulation at the tissue site 1305. In some embodiments, the tissue site 1305 may comprise a wound 1310 that extends through the epidermis 1315 and into a dermis 1320. In some examples, as shown in FIG. 13, the tissue site 1305 may comprise a wound 1310 which extends through the epidermis 1315 and dermis 1320 and into a subcutaneous tissue 1325.

In some embodiments, the dressing 110 may be applied to the tissue site 1305 and cover the wound 1310. In illustrative embodiments, the first film layer 205 may be placed within, over, on, against, or otherwise proximate to the tissue site 1305. For example, at least a portion of the bottom surface 230 of the first film layer 205 may be placed into within, over, on, against, or otherwise proximate to the wound 1310. The secondary manifold 1105 may be placed over the first film layer 205 across from the wound 1310 or epidermis 1315. For example, at least a portion of the bottom surface 1115 of the secondary manifold 1105 may be brought into contact with at least a portion of the top surface 275 of the first film layer 205. The cover 125, which may be coated on at least a portion of the bottom surface 1130 with adhesive 1330 may be positioned over the secondary manifold and the tissue interface 120 such that at least a portion of the bottom surface 1130 or adhesive 1330 is brought into contact with at least a portion of the top surface 1110 of the secondary manifold 1105 and at least a portion of the top surface 275 of the second film layer 215. In some embodiments, at least a portion of the cover 125 may be adhered to at least a portion of the secondary manifold 1105 and at least a portion of the tissue interface 120.

In some embodiments, adhesive 1330 may be present on the bottom surface 1130 of the cover 125 at the border region 1205 of the cover 125. For example, the border region 1205 of the cover 125 may be adhered to the epidermis 1315 by adhesive 1330. The cover 125 may be sealed to undamaged epidermis 1315 peripheral to the wound 1310 at least at the border region 1205. Thus, the dressing 110 may provide a sealed therapeutic environment 1335 proximate to the wound 1310. The sealed therapeutic environment 1335 may be substantially isolated from the external environment, and the negative-pressure source 105 may be fluidly coupled to the sealed therapeutic environment 1335. For example, dressing interface 1150 may be disposed over or received through the aperture 1140 formed in the cover 125. The dressing interface 1150 may for a fluid seal against the top surface 1125 of the cover 125, for example, by an adhesive seal, and the dressing interface 1150 may be in fluid communication with the sealed therapeutic environment 1335. In example embodiments, the dressing interface 1150 may be fluidly coupled to the negative-pressure source 105 by fluid conductor 1145. In illustrative embodiments, a canister, such as container 115 may be disposed in the fluid path between the dressing interface 1150 and the negative-pressure source 105. Negative pressure may be applied across the wound 1310 by through the secondary manifold 1105 and the first film layer 205 can induce macrostrain and microstrain at the wound 1310, and remove or reduce exudates and other fluids form the tissue site 1305. The removed exudates and other fluids can be collected in the container 115 and disposed of properly. In example embodiments, fluid, moisture, and exudate may travel from the wound 1310 through the fluid passages 220 in the first film layer 205 and into the windows 240, from the windows 240 through the fluid passages 270 in the second film layer 215, and through the secondary manifold 1105 and to the dressing interface 1150.

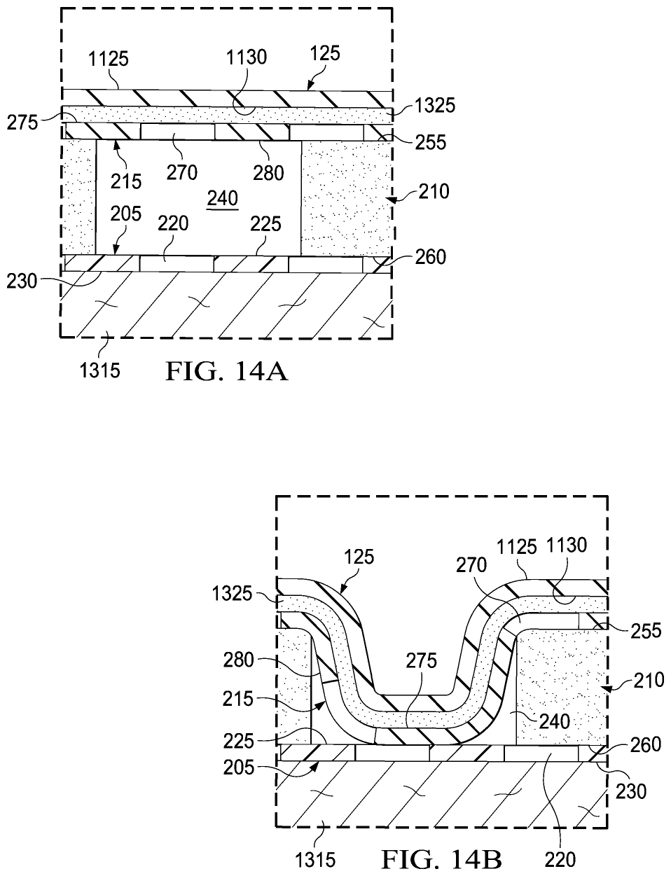

FIG. 14A is a detail view, taken at reference FIG. 14A in FIG. 13, illustrating details that may be associated with some example embodiments of the dressing 110 and system 100 of FIG. 13. FIG. 14A illustrates embodiments of the dressing 110 where the cover 125 and the second film layer 215 are not drawn into the window 240. For example, the bottom surface 280 of the second film layer 215 may remain substantially separated from the top surface 225 of the first film layer 205. In example embodiments, the second film layer 215 may be coupled to, for example, welded to at least a portion of the primary manifold 210. In examples where the second film layer 215 is welded to the primary manifold 210, the welds may substantially prevent the second film layer 215 and the cover 125 from being drawn into the window 240 under reduced pressure. In illustrative embodiments, the second film layer 215 may not be coupled to or welded to the primary manifold 210. In examples where the second film layer 215 is not welded to the primary manifold 210, the second film layer 215 may not be prevented from being drawn into the window 240. FIG. 14A may illustrate some embodiments where the second film layer 215 and the cover 125 are not drawn into the window 240, such as when negative pressure is not provided to the sealed therapeutic environment 1335. For example, the pressure within the sealed therapeutic environment 1335 may be substantially the same as the ambient pressure outside of the dressing 110, such as in the region facing the top surface 1125 of the cover 125. In cases where a pressure gradient is not created across the cover 125 and the second film layer 215, a resultant force is not created, and the cover 125 and the second film layer 215 are not drawn into the window 240.

FIG. 14B illustrates additional details that may be associated with the detail view of FIG. 14A in some embodiments of the dressing 110 and system 100 of FIG. 13. For example, the pressure within the sealed therapeutic environment 1335 may be reduced to a suitable negative pressure, resulting in a low pressure region within the primary manifold 210, such as within the window 240. In illustrative embodiments, a pressure gradient may be created across the cover 125 and the second film layer 215, with a region of higher ambient pressure opposite the top surface 1125 of the cover 125 and a region of lower negative pressure opposite the bottom surface 280 of the second film layer 215. A resultant force from the pressure differential across the cover 125 and the second film layer 215 may draw at least a portion of the cover 125 and the second film layer 215 into the window 240. For example, a portion of the bottom surface 280 of the second film layer 215 may be brought into contact with the top surface 225 of the first film layer 205. In illustrative embodiments, at least a portion of the bottom surface of the first film layer 205 may be in contact with the epidermis 1315 or the wound 1310 (not shown in FIG. 14B). In some embodiments, the cover 125, the adhesive 1330, the second film layer 215, and the first film layer 205 may be substantially clear or optically transparent, and exhibit a substantially similar refractive index. In example embodiments, the primary manifold 210 may be sufficiently stiff in a direction approximately normal to the plane formed by the top surface 1125 of the cover 125 to resist compaction or deformation under negative pressure.

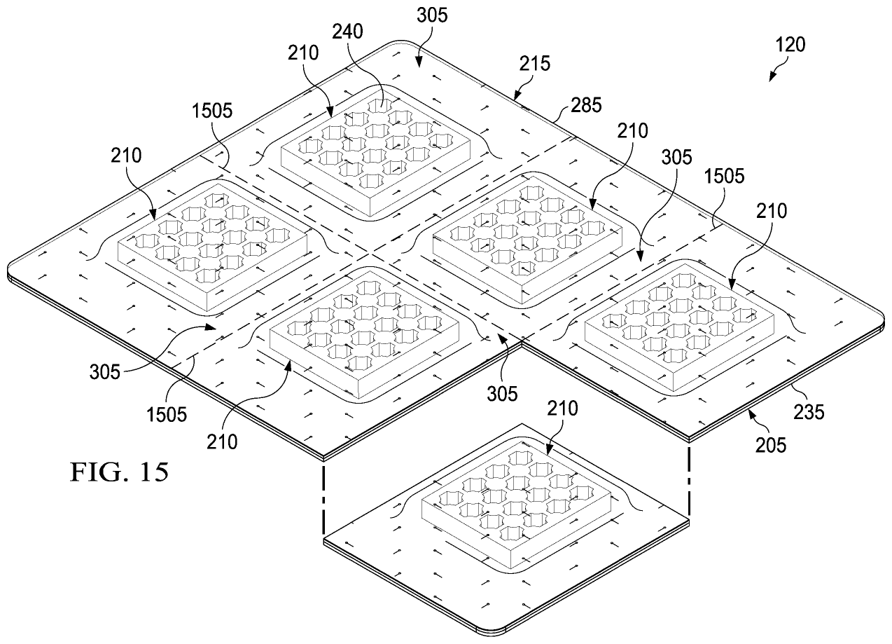

FIG. 15 is an isometric view of an assembled example of the tissue interface 120 of FIG. 2, illustrating additional details that may be associated with some embodiments. For example, a plurality of primary manifolds 210 may be disposed between the first film layer 205 and the second film layer 215. In some embodiments, a border region 305 may be formed around each of the plurality of primary manifolds 210. In illustrative embodiments, the first film layer 205 and the second film layer 215 may be perforated in the border regions 305 between the primary manifolds 210. For example, the tissue interface 120 may be resized by selectively removing one or more of the primary manifolds 210.

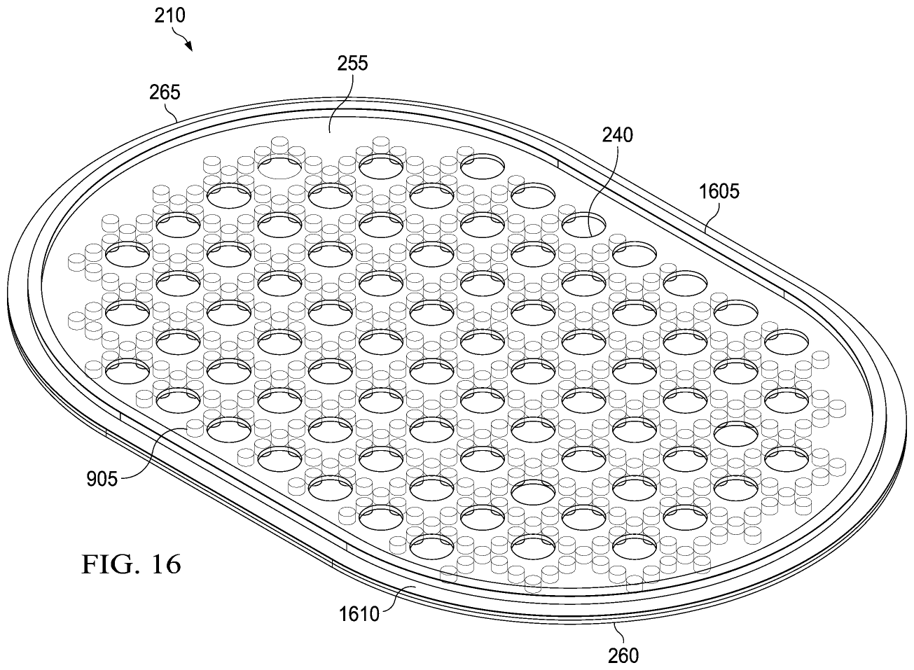

FIG. 16 is an isometric view of an example embodiment of the primary manifold 210, illustrating additional details that may be associated with the therapy system 100 of FIG. 1. For example, the primary manifold 210 may be formed as a substantially sheet-like structure comprising a top surface 255, a bottom surface 260, and a periphery 265. In some embodiments, the periphery 265 may be a stadium, discorectangle, or obround shape. The primary manifold 210 may be formed from a sheet of polyurethane, such as a vacuum-formed sheet of polyurethane with a thickness of about 0.5 mm. In example embodiments, the primary manifold 210 may be formed form a polymer material that is substantially clear or optically transparent, allowing the user to see through the primary manifold 210. As depicted in FIG. 16, accordingly to some examples, the windows 240 and the standoffs 905 may be arranged in a pattern similar to the pattern previously discussed with respect to FIG. 10A. Windows 240 may be removed from the primary manifold 210 and form a grid pattern. In some embodiments, the plurality of windows 240 may be arranged in a pattern of rows and columns. The center of each window 240 may be aligned with the center of each other window 240 within a row. The center of each window 240 may also be aligned with the center of each other window 240 within a column. In example embodiments, a plurality of standoffs 905 may be formed on the bottom surface 260 of the primary manifold 210. For example, the plurality of standoffs 905 may form a grid pattern. In illustrative embodiments, the plurality of standoffs 905 may be arranged in a pattern of rows and columns. The center of each standoff 905 within a row may be aligned with the center of each other standoff 905 within the row. The center of each standoff 905 within a column may be aligned with the center of each other standoff 905 within a column. In some embodiments, each row of the plurality of windows 240 may be disposed in between two adjacent rows of the plurality of standoffs 905. In example embodiments, each column of the plurality of windows 240 may be disposed in between two adjacent columns of the plurality of standoffs 905. In some embodiments, the pattern of the plurality of windows 240 may be arbitrarily chosen or random. In illustrative embodiments, the pattern of the plurality of standoffs 905 may be arbitrarily chosen or random.

Similar to example embodiments previously described with respect to FIG. 10A, in example embodiments, each standoff 905 may be substantially circular in profile and protrude outward in a substantially orthogonal manner from the plane of the bottom surface 260 of the primary manifold 210, and have a diameter w₃. In illustrative embodiments, each window 240 may be substantially circular in profile in the plane of the top surface 255 of the primary manifold 210, and have a diameter w₄. In example embodiments, w₃ may be substantially less than w₄. For example, w₃ may be about 3 mm in particular embodiments, and w₄ may be about 8 mm. In example embodiments, each standoff 905 within a row may be spaced a distance of about 4 mm on center from an adjacent standoff 905 within the row. In illustrative embodiments, each standoff 905 within a column may be spaced a distance of about 4 mm on center from an adjacent standoff 905 within the column. In some embodiments, windows 240 may be square, or any suitable shape. As shown in FIG. 16, example embodiments of the primary manifold 210 may also include a raised portion, such as a lip portion, a boss, or a pleat 1605. For example, the pleat 1605 may protrude outward in a substantially orthogonal manner from the plane of the top surface 255 of the primary manifold 210. In example embodiments, the pleat 1605 may have a shape similar to the periphery 265. For example, the pleat 1605, as shown in FIG. 16, in examples where the periphery 265 is a stadium, discorectangle, or obround shape, the pleat 1605 may be a scaled down stadium, discorectangle, or obround shape similar to the shape of the periphery 265. Examples of the primary manifold 210 may also include a border region 1610 between the pleat 1605 and the periphery 265. For example, the border region 1610 may be free from the windows 240 or the standoffs 905.

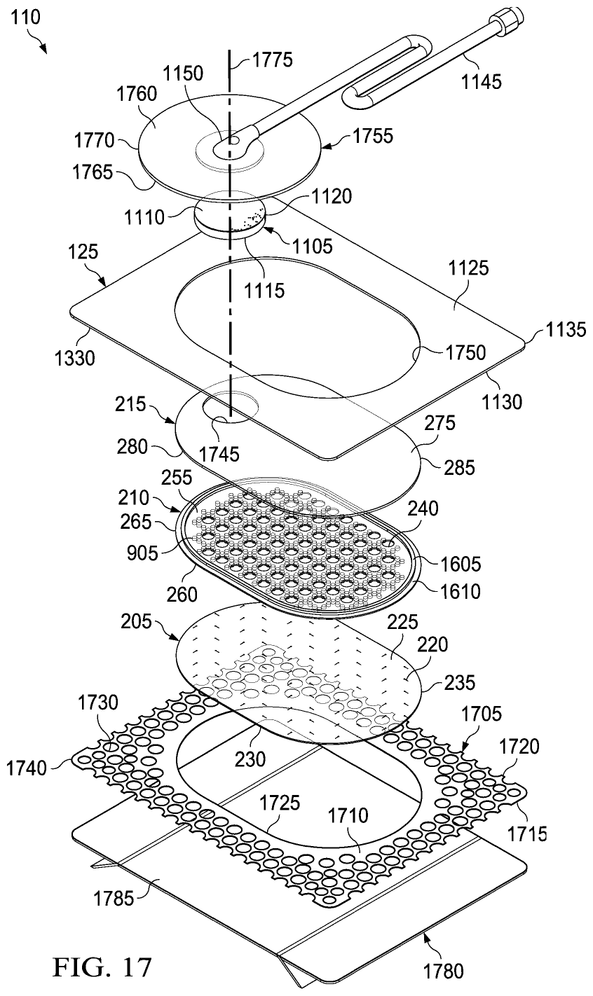

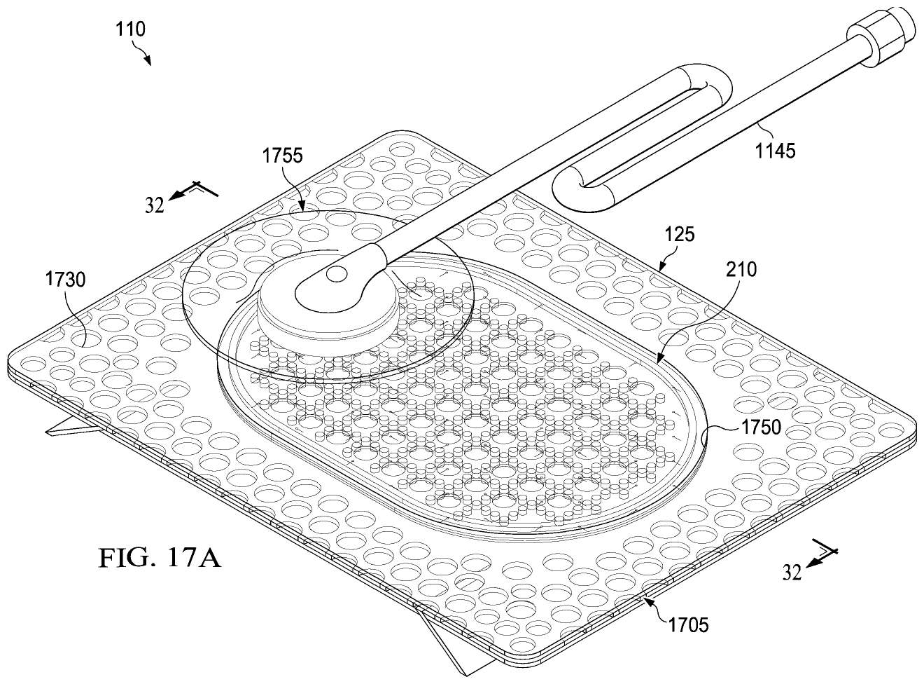

FIG. 17 is an exploded view of an example of the dressing 110 of FIG. 1, illustrating additional details that may be associated with some embodiments. In the example of FIG. 17, the dressing 110 comprises a sealing layer 1705, the first film layer 205, the primary manifold 210, the second film layer 215, the cover 125, the secondary manifold 1105, and the dressing interface 1150. In some embodiments, the sealing layer 1705 may be formed from a soft, pliable material suitable for providing a fluid seal with a tissue site, such as a suitable gel material, and may have a substantially flat surface. The sealing layer 1705 may include, without limitation, a silicone gel, a soft silicone, hydrocolloid, hydrogel, polyurethane gel, polyolefin gel, hydrogenated styrenic copolymer gel, a foamed gel, a soft closed-cell foam such as polyurethanes and polyolefins coated with an adhesive, polyurethane, polyolefin, or hydrogenated styrenic copolymers. In some embodiments, the sealing layer 1705 may have a thickness between about 200 micrometers and about 1,000 micrometers. Further, the sealing layer 1705 may be formed from hydrophobic or hydrophilic materials.

In some embodiments, the sealing layer 1705 may be a hydrophobic-coated material. For example, the sealing layer 1705 may be formed by coating a spaced material, such as, for example, woven, nonwoven, molded, or extruded mesh with a hydrophobic material. The hydrophobic material for the coating may be a soft silicone, for example.

The sealing layer 1705 may have a top surface 1710 opposite a bottom surface 1715, a periphery 1720 defined by the outer perimeter of the sealing layer 1705, and a treatment aperture 1725 formed through the sealing layer 1705. The treatment aperture 1725 may have an outline complementary to or corresponding to the periphery 265 of the primary manifold 210 in some example. For example, the treatment aperture 1725 may form a frame, window, or other opening around a surface, such as the border region 1610 of the primary manifold 210. The sealing layer 1705 may also have a plurality of apertures 1730 formed through the sealing layer 1705 in the region of the sealing layer 1705 defined between the treatment aperture 1725 and the periphery 1720 of the sealing layer 1705. The sealing layer 1705 may have an interior border 1735 around the treatment aperture 1725, which may be substantially free of the apertures 1730. In some examples, as illustrated in FIG. 17, the treatment aperture 1725 may have a shape similar to the periphery 265 of the primary manifold 210, and may be symmetrical and centrally disposed in the sealing layer 1705, forming an open central window. For example, the treatment aperture 1725 may have a shape similar to the stadium, discorectangle, or obround shape of the periphery 265, scaled to be smaller.

The apertures 1730 may be formed by cutting, perforating, or by application of local RF or ultrasonic energy, for example, or by other suitable techniques for forming an opening or perforation in the sealing layer 1705. The apertures 1730 may have a uniform distribution pattern, or may be randomly distributed on the sealing layer 1705. The apertures 1730 formed through the sealing layer 1705 may have many shapes, including circles, squares, stars, ovals, polygons, slits, complex curves, rectilinear shapes, triangles, for example, or may have some combination of such shapes.

Each of the apertures 1730 may have uniform or similar geometric properties. For example, in some embodiments, each of the apertures 1730 may be circular apertures, having substantially the same diameter. In some embodiments, each of the apertures 1730 may have a diameter of about 1 millimeter to about 50 millimeters. In other embodiments, the diameter of each of the apertures 1730 may be about 1 millimeter to about 20 millimeters.

In some embodiments, the geometric properties of the apertures 1730 may vary. For example, the diameter of the apertures 1730 may vary depending on the position of the apertures 1730 in the sealing layer 1705. For example, some of the apertures 1730 may have a diameter between about 5 millimeters and about 10 millimeters. A range of about 7 millimeters to about 9 millimeters may be suitable for some examples. In some embodiments, the apertures 1730 disposed at or near the corners 1740 of the sealing layer 1705 may have a diameter between about 7 millimeters and about 8 millimeters.

At least one of the apertures 1730 may be positioned near the periphery 1720 of the sealing layer 1705, and may have an interior cut open or exposed at the periphery 1720 that is in lateral communication in a lateral direction with the periphery 1720. The lateral direction may refer to a direction towards the periphery 1720 and in a same plane as the sealing layer 1705. As shown in the example of FIG. 17, the apertures 1730 in the region between the treatment aperture 1725 and the periphery 1720 of the sealing layer 1705 may be positioned proximate to or at the periphery 1720 and in fluid communication in a lateral direction with the periphery 1720. The apertures 1730 positioned proximate to or at the periphery 1720 may be spaced substantially equidistant around the periphery 1720 as shown in the example of FIG. 17. Alternatively, the spacing of the apertures 1730 proximate to or at the periphery 1720 may be irregular.

As shown in FIG. 17, according to some embodiments of the dressing 110, the first film layer 205 may have a periphery 235 which is coextensive with the periphery 265 of the primary manifold 210. The second film layer 215 may have a periphery 285 which is coextensive with the periphery 265 of the primary manifold 210. In some examples, as illustrated in FIG. 17, the second film layer 215 may include an aperture 1745 formed through the second film layer 215, but the second film layer 215 may otherwise be solid and free of other apertures or passages. For example, the second film layer 215 in the example of FIG. 17 may not include any fluid passages 270 shown in other examples, such as those in FIG. 2. In assembled form, a portion of the top surface 225 of the first film layer 205 may be coupled, such as by RF welding, to a portion of the bottom surface 260 of the primary manifold 210. For example, a portion of the top surface 225 near the periphery 235 may be coupled to a portion of the bottom surface 260 near the periphery 265 of the primary manifold 210, such as at the border region 1610. In assembled form, a portion of the bottom surface 280 of the second film layer 215 may be coupled, such as by RF welding, to a portion of the top surface 255 of the primary manifold 210. For example, a portion of the bottom surface 280 near the periphery 285 may be coupled to a portion of the top surface 255 near the periphery 265 of the primary manifold 210, such as at the border region 1610.

As illustrated in FIG. 17, examples of the dressing 110 may include a cover 125 with a central aperture 1750 formed through the cover 125. A perimeter of the central aperture 1750 may be substantially coextensive with the perimeter of the treatment aperture 1725. The shape of the perimeters of the treatment aperture 1725 and the central aperture 1750 may be similar to the shape of the periphery 235, periphery 265, and periphery 285. The perimeters of the treatment aperture 1725 and the central aperture 1750 may be scaled to bound an area smaller than the area bound by the periphery 235, periphery 265, and periphery 285. For example, in assembled form, a portion of the bottom surface 230 of the first film layer 205 near the periphery 235 may be coupled or adhered to a portion of the top surface 1710 of the sealing layer 1705 near the treatment aperture 1725. In assembled form, a portion of the bottom surface 1130 of the cover 125 near the central aperture 1750 may be coupled or adhered to a portion of the top surface 275 of the second film layer 215 near the periphery 285, such as by the adhesive 1330 disposed on the bottom surface 1130 of the cover 125. A portion of the bottom surface 1130 of the cover 125 may be coupled or adhered to a portion of the top surface 1710 of the sealing layer 1705, such as by the adhesive 1330 disposed on the bottom surface 1130 of the cover 125.

Some examples of the dressing 110 may also include the secondary manifold 1105 and the dressing interface 1150. In example embodiments, the dressing interface 1150 may also include a connector drape 1755. The connector drape 1755 may include a top surface 1760, a bottom surface 1765, a periphery 1770, and be formed from a material similar to the material of the cover 125. The bottom surface 1765 may be coated with an adhesive, and a portion of the bottom surface 1765 may be adhered to a portion of the dressing interface 1150. In assembled form, a centroid of the aperture 1745 may be aligned with a centroid of the secondary manifold 1105, a centroid of the dressing interface 1150, and a centroid of the connector drape 1755 along an axis 1775. The axis 1775 may be substantially normal to the planes defined by the top surface 1710, top surface 225, top surface 255, top surface 275, top surface 1125, and/or top surface 1110. In assembled form, a portion of the bottom surface 1115 of the secondary manifold 1105 near the periphery 1120 may be in contact with a portion of the top surface 275 of the second film layer 215 and/or a portion of the top surface 1125 of the cover 125 around the aperture 1745. The dressing interface 1150 may be disposed adjacent to the top surface 1110 of the secondary manifold 1105 and be in fluid communication with the secondary manifold 1105. The bottom surface 1765 of the connector drape 1755 may be coupled or adhered to a portion of the connector 1170, the top surface 1110 of the secondary manifold 1105, the top surface 1125 of the cover 125, and/or the top surface 275 of the second film layer 215.

As illustrated in the example of FIG. 17, in some embodiments, the dressing 110 may include a release liner 1780 to protect the sealing layer 1705 and the adhesive 1330 prior to use. The release liner 1780 may also provide stiffness to assist with, for example, deployment of the dressing 110. The release liner 1780 may be, for example, a casting paper, a film, or polyethylene. Further, in some embodiments, the release liner 1780 may be a polyester material such as a polyethylene terephthalate (PET), or similar polar semi-crystalline polymer. The use of a polar semi-crystalline polymer for the release liner 1780 may substantially preclude wrinkling or other deformation of the dressing 110. For example, the polar semi-crystalline polymer may be highly orientated and resistant to softening, swelling, or other deformation that may occur when objects are brought into contact with the layers and/or components of the dressing 110, or when the dressing 110 is subjected to temperature or environmental variations, or during sterilization. Further, a release agent may be disposed on a top surface 1785 of the release liner 1780 that is configured to contact the bottom surface 1715 of the sealing layer 1705 and the adhesive 1330. For example, the release agent may be a silicone coating and may have a release factor suitable to facilitate removal of the release liner 1780 by hand and without damaging or deforming the dressing 110. In some embodiments, the release agent may be a fluorocarbon or a fluorosilicone, for example. In other embodiments, the release liner 1780 may be uncoated or otherwise used without a release agent.

FIG. 17A is an isometric view of an assembled example of the dressing 110 of FIG. 17. As shown in the example of FIG. 17A, the connector drape 1755, the cover 125, the second film layer 215, and/or the first film layer 205 may be substantially clear or optically transparent, allowing for visualization of the layers of the dressing 110 as well as visualization through the windows 240 of the primary manifold 210 of the dressing 110.

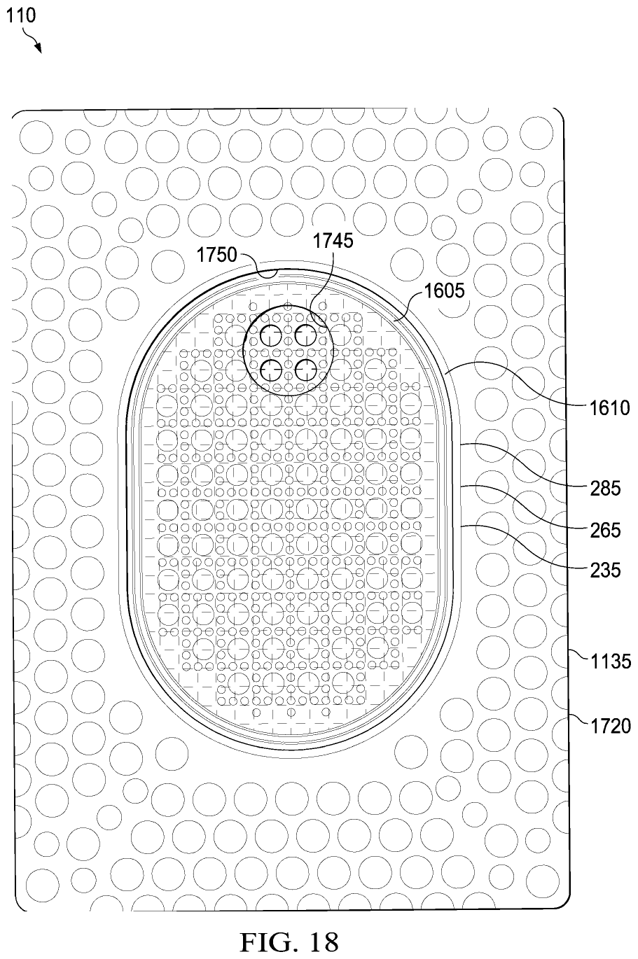

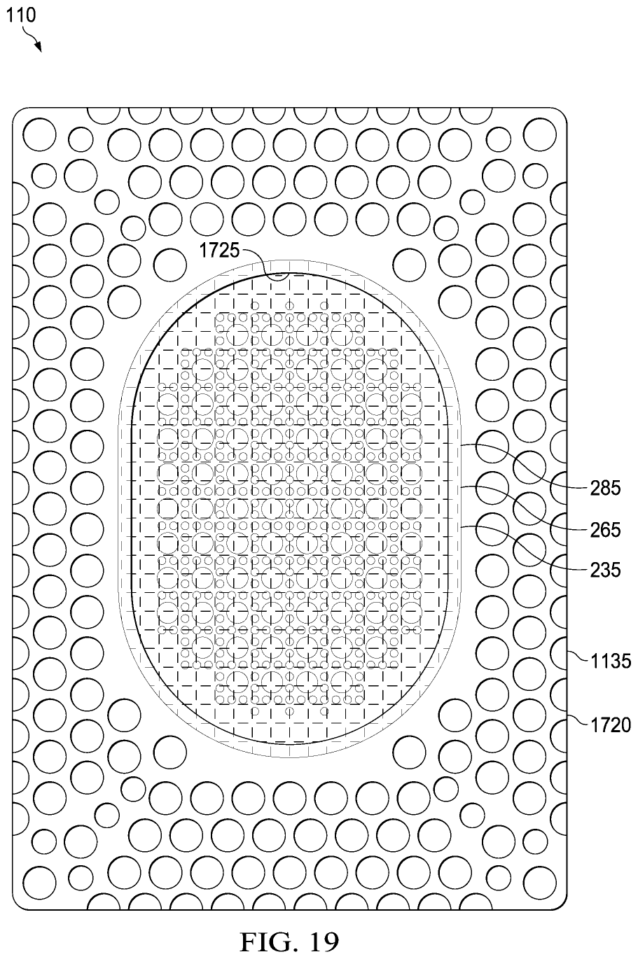

FIG. 18 is a top view of the dressing 110 of FIG. 17, as assembled, illustrating details that may be associated with some embodiments. FIG. 19 is a bottom view of the dressing 110 of FIG. 17, as assembled, illustrating details that may be associated with some embodiments. As illustrated in FIGS. 18 and 19, in some examples of the dressing 110, the periphery 1135 of the cover 125 may be coextensive with the periphery 1720 of the sealing layer 1705. The periphery 235 of the first film layer 205 may be coextensive with the periphery 265 of the primary manifold 210 and the periphery 285 of the second film layer 215. The perimeter of the central aperture 1750 may be coextensive with the perimeter of the treatment aperture 1725 in a plane defined by the top surface 1125 of the cover 125 or the bottom surface 1715 of the sealing layer 1705. As previously described with respect to FIG. 17, the shape of the perimeters of the treatment aperture 1725 and of the central aperture 1750 may be similar to the periphery 235, periphery 265, and periphery 285, but scaled down such that in the assembled form, a portion of the sealing layer 1705 around the treatment aperture 1725 overlaps with a portion of the first film layer 205 around the periphery 235, and a portion of the cover 125 around the central aperture 1750 overlaps with a portion of the second film layer 215 around the periphery 285.

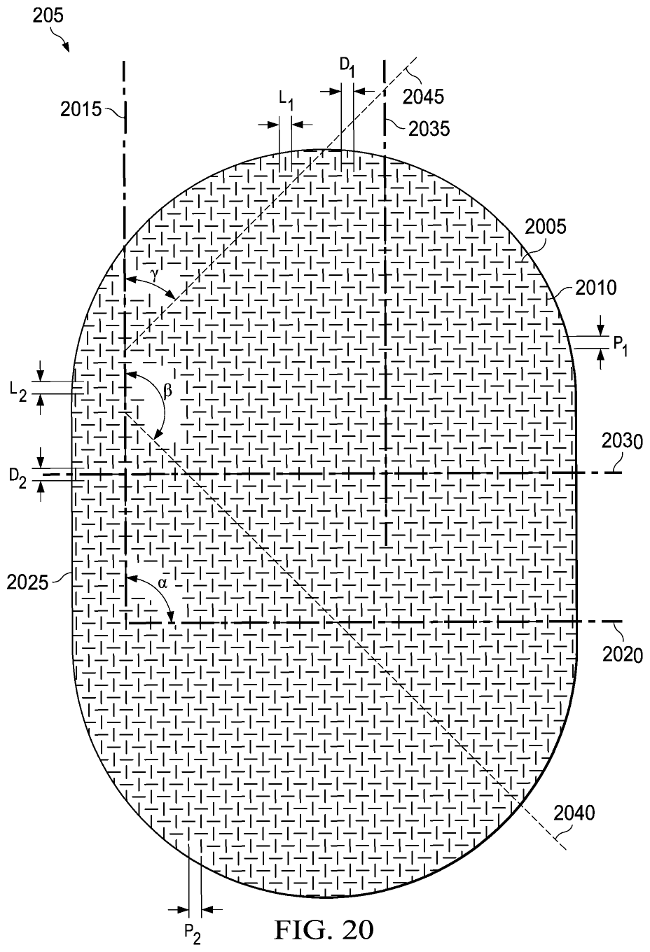

FIGS. 20-27 are top views illustrating additional details that may be associated with some embodiments of the first film layer 205. For example, as illustrated in FIG. 17, the fluid passages 220 may include a first plurality of perforations 2005 and a second plurality of perforations 2010. Each of the first plurality of perforations 2005 and the second plurality of perforations 2010 may be linear or curved perforations, such as slots or slits. In some embodiments where the perforations are linear slots or slits, each of the first plurality of perforations 2005 may have a length L₁ and each of the second plurality of perforations 2010 may have a length L₂. In some embodiments, where the perforations are curved slots or slits, each of the first plurality of perforations may have a length L₁ measured from an end of the curved slot or slit to the other end of the curved slot or slit, and each of the second plurality of perforations may have a length L₂ measured from an end of the curved slot or slit to the other end of the curved slot or slit. In some embodiments, the length L₁ may be equal to the length L₂. The first plurality of perforations 2005 and the second plurality of perforations 2010 may be distributed across the second layer in one or more rows in one direction or in different directions.

In example embodiments, each of the first plurality of perforations 2005 may have a first long axis. In some embodiments, the first long axis may be parallel to a first reference line 2015 running in a first direction. In illustrative examples, each of the second plurality of perforations 2010 may have a second long axis. In example embodiments, the second long axis may be parallel to a second reference line 2020 running in a second direction. In some embodiments, one or both of the first reference line 2015 and the second reference line 2020 may be defined relative to an edge 2025 or line of symmetry of the first film layer 205. For example, one or both of the first reference line 2015 and the second reference line 2020 may be parallel or coincident with an edge 2025 or line of symmetry of the first film layer 205. In some illustrative embodiments, one or both of the first reference line 2015 and the second reference line 2020 may be rotated an angle relative to an edge 2025 of the first film layer 205. In example embodiments, an angle α may define the angle between the first reference line 2015 and the second reference line 2020.

In some example embodiments, the centroid of each of the first plurality of perforations 2005 within a row may intersect a third reference line 2030 running in a third direction. In illustrative embodiments, the centroid of each of the second plurality of perforations 2010 within a row may intersect a fourth reference line 2035 running in a fourth direction. In general, a centroid refers to the center of mass of a geometric object. In the case of a substantially two dimensional object such as a linear slit, the centroid of the linear slit will be the midpoint.

The pattern of fluid passages 220 may also be characterized by a pitch, which indicates the spacing between corresponding points on fluid passages 220 within a pattern. In example embodiments, pitch may indicate the spacing between the centroids of fluid passages 220 within the pattern. Some patterns may be characterized by a single pitch value, while others may be characterized by at least two pitch values. For example, if the spacing between centroids of the fluid passages 220 is the same in all orientations, the pitch may be characterized by a single value indicating the spacing between centroids in adjacent rows. In example embodiments, a pattern comprising a first plurality of perforations 2005 and a second plurality of perforations 2010 may be characterized by two pitch values, P₁ and P₂, where P₁ is the spacing between the centroids of each of the first plurality of perforations 2005 in adjacent rows, and P₂ is the spacing between the centroids of each of the second plurality of perforations 2010 in adjacent rows.

In example embodiments, within each row of the first plurality of perforations 2005, each perforation may be separated from an adjacent perforation by a distance D₁. In some embodiments, within each row of the second plurality of perforations 2010, each perforation may be separated from an adjacent perforation by a distance D₂. In some patterns, the rows may be staggered. The stagger may be characterized by an orientation of corresponding points in successive rows relative to an edge or other reference line associated with the first film layer 205. In some embodiments, the rows of the first plurality of perforations 2005 may be staggered. For example, a fifth reference line 2040 in a fifth direction runs through the centroids of corresponding perforations of adjacent rows of the first plurality of perforations 2005. In some example embodiments, the stagger of the rows of the first plurality of perforations 2005 may be characterized by the angle ft formed between the first reference line 2015 and the fifth reference line 2040. In additional illustrative embodiments, the rows of the second plurality of perforations 2010 may also be staggered. For example, a sixth reference line 2045 in a sixth direction runs through the centroids of corresponding perforations of adjacent rows of the second plurality of perforations 2010. In some embodiments, the stagger of the rows of the second plurality of perforations 2010 may be characterized by the angle γ formed between the first reference line 2015 and the sixth reference line 2045.

FIG. 20 illustrates an example of a pattern that may be associated with some embodiments of the fluid passages 220. In the example of FIG. 20, each of the first plurality of perforations 2005 and the second plurality of perforations 2010 may be linear slots or slits. The first reference line 2015 may be parallel with an edge 2025, and the second reference line 2020 may be orthogonal to the edge 2025. In example embodiments, the third reference line 2030 is orthogonal to the first reference line 2015, and the fourth reference line 2035 is orthogonal to the second reference line 2020. For example, the third reference line 2030 may be incident with the centroids of corresponding perforations in alternating rows of the second plurality of perforations 2010, and the fourth reference line 2035 may intersect the centroids of corresponding perforations in alternating rows of the first plurality of perforations 2005. In the example of FIG. 20, the fluid passages 220 are arranged in a cross-pitch pattern in which each of the first plurality of perforations 2005 is orthogonal along its first long axis to each of the second plurality of perforations 2010 along its second long axis. For example, in FIG. 20, P₁ is equal to P₂ (within acceptable manufacturing tolerances), and the cross-pitch pattern may be characterized by a single pitch value. Additionally, L₁ and L₂ may be substantially equal, and D₁ and D₂ may be also be substantially equal, all within acceptable manufacturing tolerances. The rows of the first plurality of perforations 2005 and the rows of the second plurality of perforations 2010 may be characterized as staggered. For example, in some example embodiments illustrated, α may be about 90°, β may be about 135°, γ may be about 45°, P₁ may be about 4 mm, P₂ may be about 4 mm, L₁ may be about 3 mm, L₂ may be about 3 mm, D₁ may be about 5 mm, and D₂ may be about 5 mm.

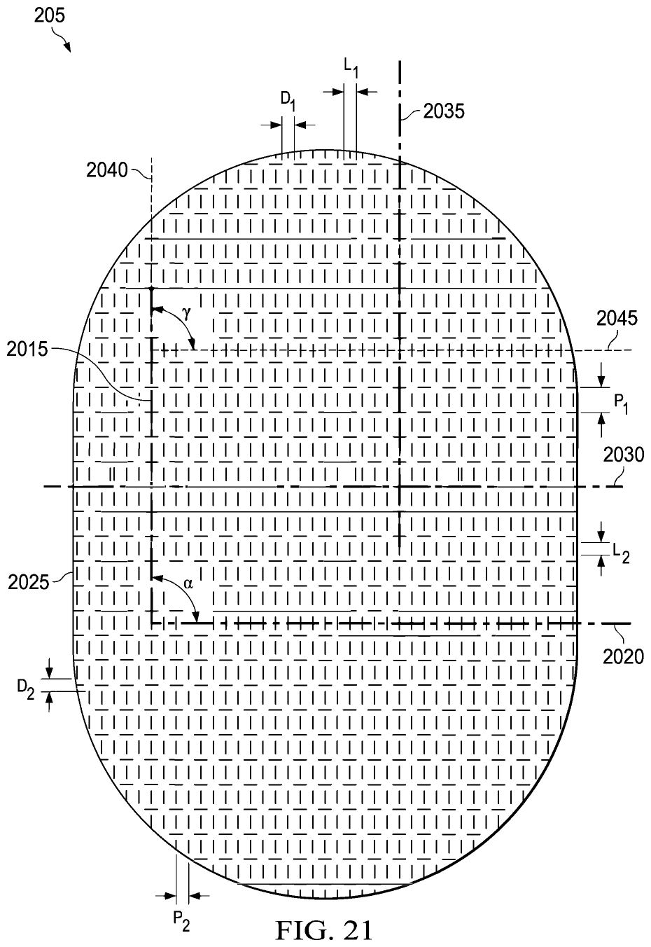

FIG. 21 is a schematic diagram of another example pattern that may be associated with some illustrative embodiments of the fluid passages 220. In illustrative examples of FIG. 21, each of the first plurality of perforations 2005 and the second plurality of perforations 2010 may be linear slits. The first reference line 2015 may be parallel with the edge 2025, and the second reference line 2020 may be orthogonal to the edge 2025. In some example embodiments, the third reference line 2030 is orthogonal to the first reference line 2015, and the fourth reference line 2035 is orthogonal to the second reference line 2020. In the example of FIG. 21, the third reference line 2030 does not intersect or touch any of the second plurality of perforations 2010, and the fourth reference line 2035 may intersect the centroids of corresponding perforations in alternating rows of the first plurality of perforations 2005. In example embodiments, the third reference line 2030 may be equidistant from the centroids of corresponding adjacent perforations within each row of the second plurality of perforations 2010. The pattern of FIG. 21 may also be characterized as a cross-pitch pattern, in which P₁ is not equal to P₂. In the example of FIG. 21, P₁ is larger than P₂. Additionally, L₁, L₂, D₁, and D₂ are substantially equal in the example of FIG. 21. In some embodiments, α may be about 90°, β may be about 0° such that the first reference line 2015 is coincident with the fifth reference line 740, γ may be about 90°, P₁ may be about 6 mm, P₂ may be about 3 mm, L₁ may be about 3 mm, L₂ may be about 3 mm, D₁ may be about 3 mm, and D₂ may be about 3 mm.

FIG. 22 illustrates an additional example of a pattern that can be associated with some embodiments of the fluid passages 220. In the example of FIG. 22, each of the first plurality of perforations 2005 and the second plurality of perforations 2010 may be linear slits. The first reference line 2015 may be parallel with an edge 2025, and the second reference line 2020 may be orthogonal to an edge 2025. In example embodiments, the third reference line 2030 is orthogonal to the first reference line 2015, and the fourth reference line 2035 is orthogonal to the second reference line 2020. In the example of FIG. 22, the third reference line 2030 does not intersect or touch any of the second plurality of perforations 2010, and the fourth reference line 2035 does not intersect or touch any of the first plurality of perforations 2005. In example embodiments, the third reference line 2030 may be equidistant from the centroids of corresponding adjacent perforations within each row of the second plurality of perforations 2010, and the fourth reference line 2035 may be equidistant from the centroids of corresponding adjacent perorations within each row of the first plurality of perforations 2005. The pattern of FIG. 22 may be characterized as a cross-pitch pattern, in which P₁ is substantially equal to P₂. Additionally, L₁, L₂, D₁, and D₂ are substantially equal in the example of FIG. 22. In some embodiments, α may be about 90°, β may be about 0° such that the first reference line 2015 is coincident with the fifth reference line 740, γ may be about 90°, P₁ may be about 6 mm, P₂ may be about 6 mm, L₁ may be about 3 mm, L₂ may be about 3 mm, D₁ may be about 3 mm, and D₂ may be about 3 mm.

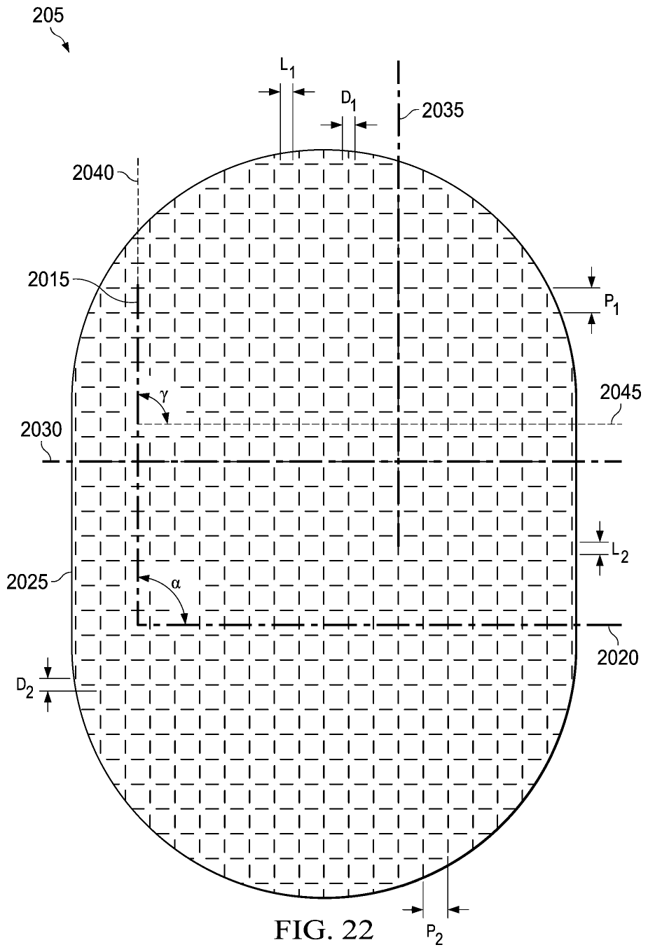

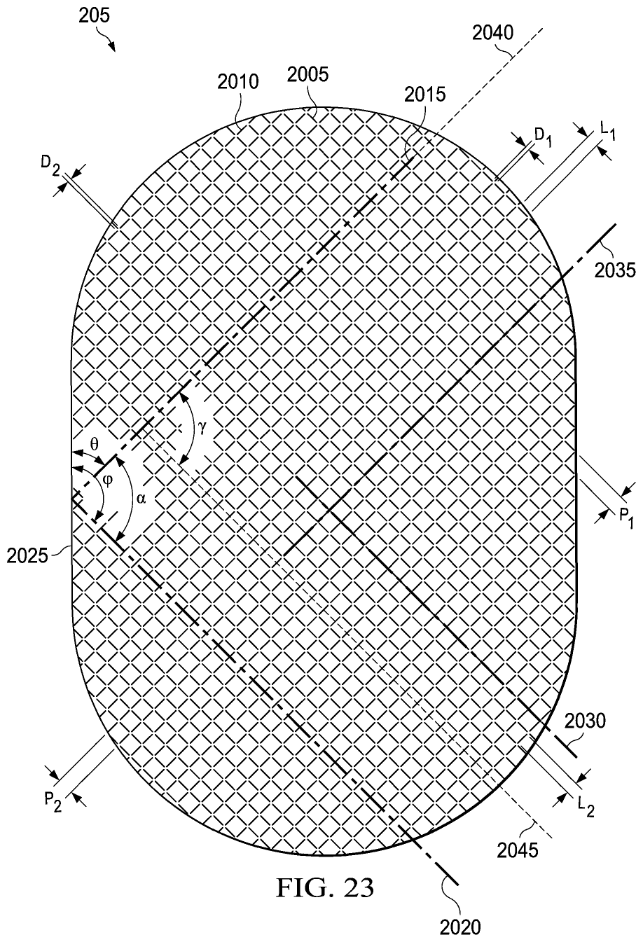

FIG. 23 illustrates additional embodiments of a pattern that may be associated with some embodiments of the fluid passages 220. In the example of FIG. 23, each of the first plurality of perforations 2005 and the second plurality of perforations 2010 may be linear slits. The first reference line 2015 may form an angle θ with an edge 2025, and the second reference line 2020 may form an angle φ an edge 2025. In example embodiments, the third reference line 2030 is orthogonal to the first reference line 2015, and the fourth reference line 2035 is orthogonal to the second reference line 2020. In the example of FIG. 23, the third reference line 2030 does not intersect or touch any of the second plurality of perforations 2010, and the fourth reference line 2035 does not intersect or touch any of the first plurality of perforations 2005. In example embodiments, the third reference line 2030 may be equidistant from the centroids of corresponding adjacent perforations within each row of the second plurality of perforations 2010, and the fourth reference line 2035 may be equidistant from the centroids of corresponding adjacent perorations within each row of the first plurality of perforations 2005. The pattern of FIG. 23 may be characterized as a cross-pitch pattern, in which P₁ is substantially equal to P₂. Additionally, L₁ may be substantially equal to L₂, and D₁ may be substantially equal to D₂ in the example of FIG. 23. In some embodiments, β may be about 0° such that the first reference line 2015 is coincident with the fifth reference line 2040, γ may be about 90°, θ may be about 45°, and φ may be about 135°.

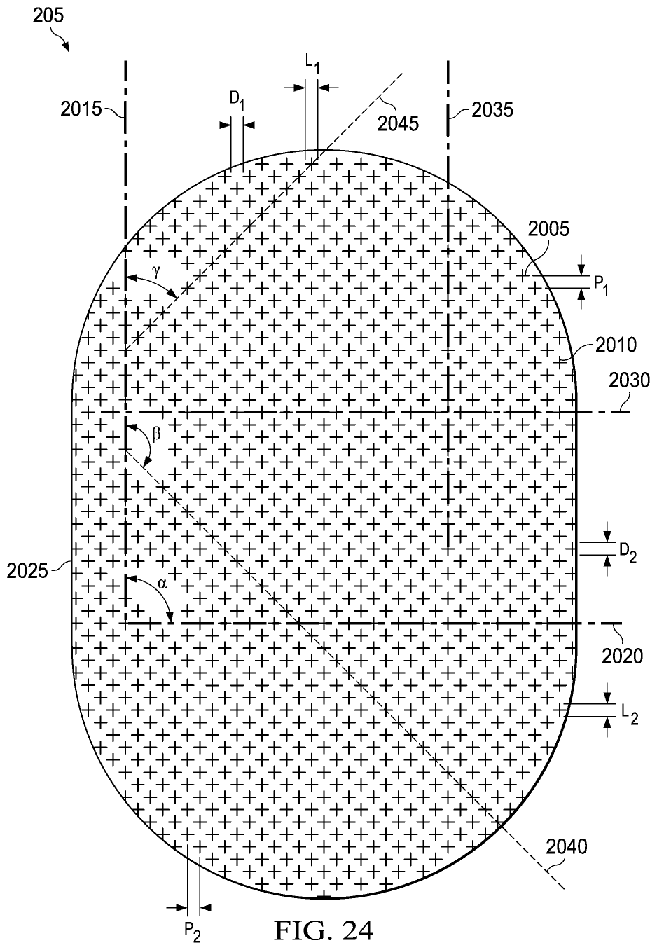

FIG. 24 illustrates examples that may be associated with some embodiments of the fluid passages 220. In some embodiments of FIG. 24, each of the first plurality of perforations 2005 and the second plurality of perforations 2010 may be linear slits. The first reference line 2015 may be parallel with an edge 2025, and the second reference line 2020 may be orthogonal to an edge 2025. In example embodiments, the third reference line 2030 is orthogonal to the first reference line 2015, and the fourth reference line 2035 is orthogonal to the second reference line 2020. For example, the third reference line 2030 may be incident with the centroids of corresponding perforations in alternating rows of the second plurality of perforations 2010, and the fourth reference line 2035 may be incident with the centroids of corresponding perforations in alternating rows of the first plurality of perforations 2005. In the example of FIG. 24, the centroid of each perforation of the first plurality of perforations 2005 is incident with the centroid of a perforation of the second plurality of perforations 2010. The fluid passages 220 are arranged in a cross-pitch pattern in which each of the first plurality of perforations 2005 is orthogonal along its first long axis to each of the second plurality of perforations 2010 along its second long axis. For example, in FIG. 24, P₁ is substantially equal to P₂, and the cross-pitch pattern may be characterized by a single pitch value. Additionally, L₁ and L₂ may be substantially equal, and D₁ and D₂ may be also be substantially equal, all within acceptable manufacturing tolerances. The rows of the first plurality of perforations 2005 and the rows of the second plurality of perforations 2010 may be characterized as staggered. In some example embodiments of FIG. 24, α may be about 90°, β may be about 135°, γ may be about 45°.

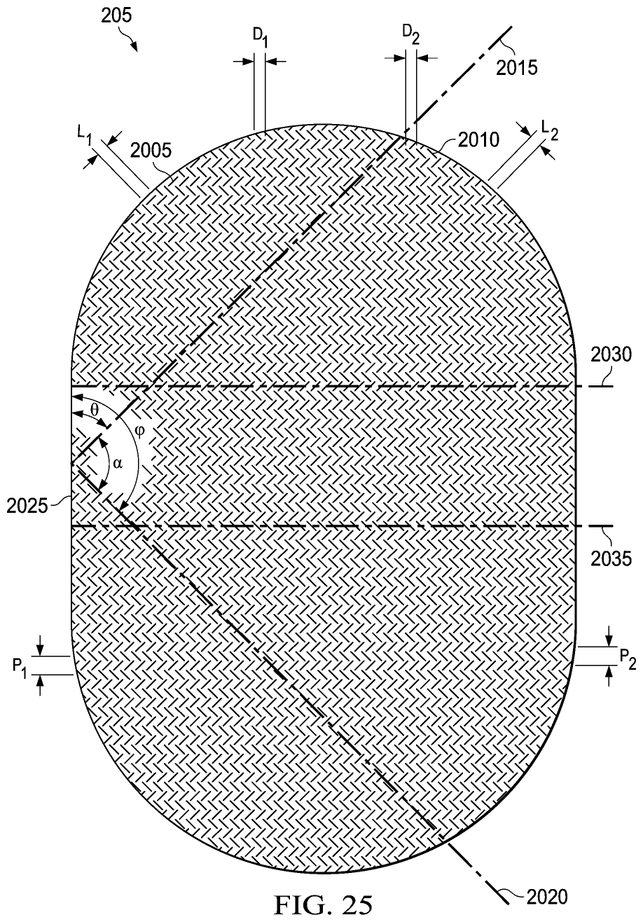

FIG. 25 show additional embodiments associated with certain illustrative embodiments of the fluid passages 220. In the example of FIG. 25, each of the first plurality of perforations 2005 and the second plurality of perforations 2010 may be linear slits. The first reference line 2015 may form an angle θ with an edge 2025. The second reference line 2020 may form an angle φ with an edge 2025. In example embodiments of FIG. 25, the third reference line 2030 and the fourth reference line 2035 may be orthogonal to an edge 2025. In the example of FIG. 25, the rows of the first plurality of perforations 2005 and the rows of the second plurality of perforations 2010 may be characterized as mirrored rows running in one direction parallel with an edge 2025 of the first film layer 205. For example, L₁ and L₂ may be substantially equal, D₁ and D₂ may be substantially equal, and P₁ and P₂ may be substantially equal, within acceptable manufacturing tolerances. In some embodiments, θ may be about 45°, and φ may be about 135°. The pattern of FIG. 25 may be characterized as a herringbone pattern.

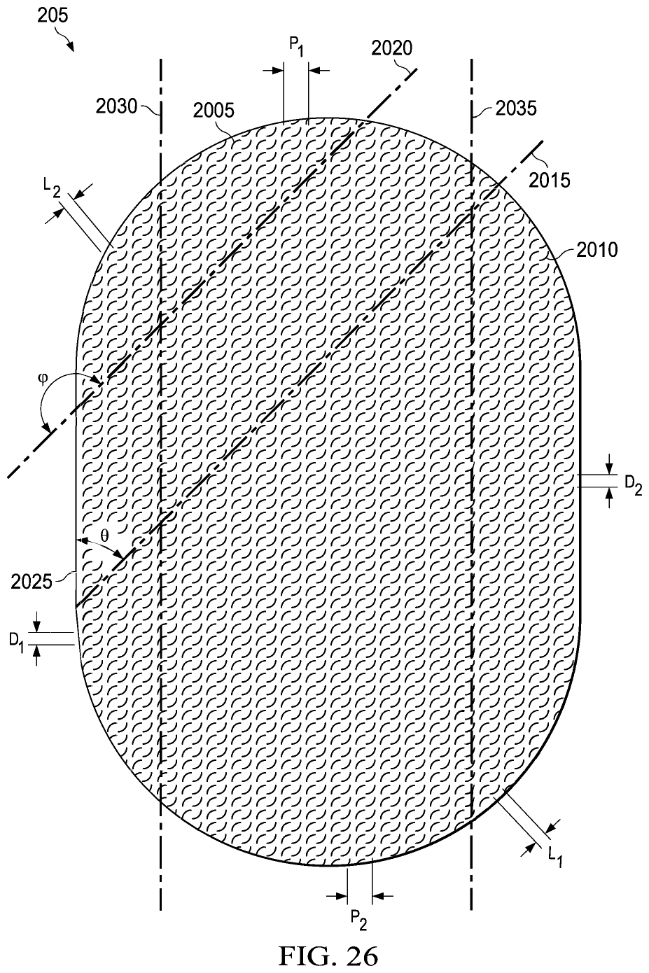

FIG. 26 show additional example embodiments associated with certain illustrative embodiments of the fluid passages 220. In the example of FIG. 26, each of the first plurality of perforations 2005 and the second plurality of perforations 2010 may be curved slits. The first reference line 2015 may form an angle θ with an edge 2025. The second reference line 2020 may form an angle φ with an edge 2025. In example embodiments of FIG. 26, the third reference line 2030 and the fourth reference line 2035 may be parallel to an edge 2025. In the example of FIG. 26, the rows of the first plurality of perforations 2005 and the rows of the second plurality of perforations 2010 may be characterized as mirrored rows running in one direction parallel with an edge 2025 of the first film layer 205. The rows of the first plurality of perforations 2005 and the rows of the second plurality of perforations 2010 may be characterized as in an embodiment of FIG. 26. For example, L₁ and L₂ may be substantially equal, D₁ and D₂ may be substantially equal, and P₁ and P₂ may be substantially equal, within acceptable manufacturing tolerances. In some embodiments, θ may be about 45°, and φ may be about 225°.

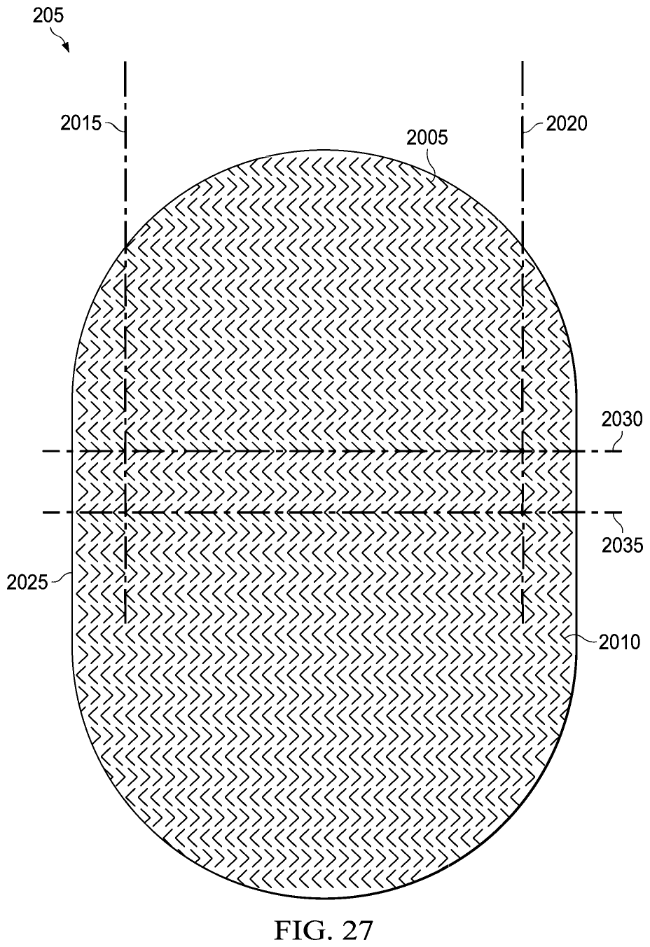

FIG. 27 shows additional embodiments associated with certain embodiments of the fluid passages 220. In the example of FIG. 27, each of the first plurality of perforations 2005 and the second plurality of perforations 2010 may be characterized as chevron slits. Each chevron slit may be formed from two orthogonal linear slits of the same length coincident at an endpoint. The chevron slit may be characterized as pointing in the direction defined by the vector drawn from the centroid of the chevron slit to the coincident endpoints. Within each row of the first plurality of perforations 2005, the chevron slits point in the same direction. Within each row of the second plurality of perforations 2010, the chevron slits point in the same direction. In example embodiments, the chevron slits of the first plurality of perforations 2005 and the chevron slits of the second plurality of perforations 2010 point in opposite directions. In example embodiments, the first reference line 2015 and the second reference line 2020 may be parallel with an edge 2025. In illustrative embodiments, the third reference line 2030 and the fourth reference line 2035 may be orthogonal to the first reference line 2015. In the example of FIG. 27, the rows of the first plurality of perforations 2005 and the rows of the second plurality of perforations 2010 may be characterized as mirrored rows running in one direction orthogonal to an edge 2025 of the first film layer 205.

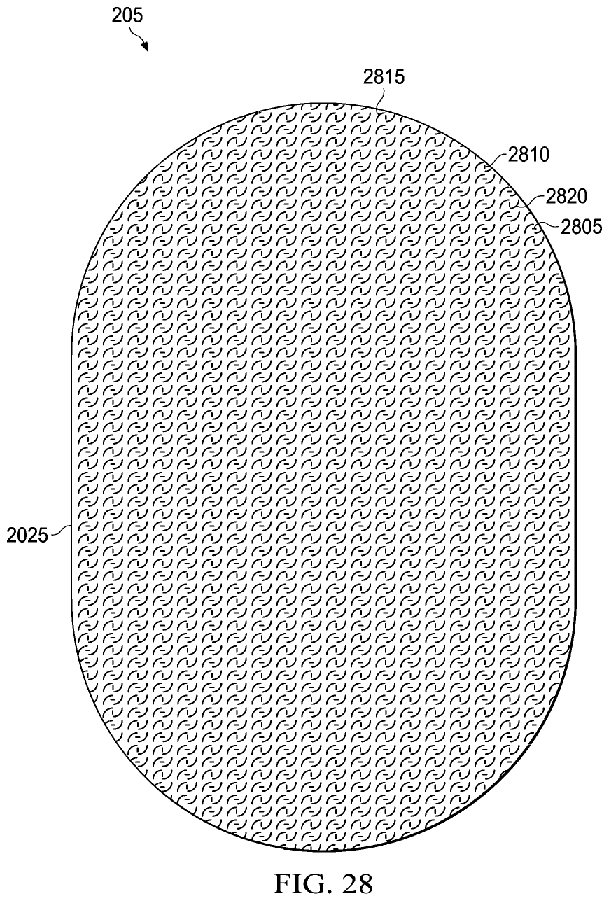

FIG. 28 further illustrates example embodiments that may be associated with some embodiments of the fluid passages 220. Certain patterns of the fluid passages 220 may comprise a third plurality of perforations 2805, a fourth plurality of perforations 2810, a fifth plurality of perforations 2815, and a sixth plurality of perforations 2820. Each of the third plurality of perforations 2805 may be a linear slit substantially orthogonal along a long axis to the edge 2025. Each of the fourth plurality of perforations 2810 may be a linear slit substantially orthogonal to the long axis of third plurality of perforations 2805 along a long axis. Each of the fifth plurality of perforations 2815 may be a curved slit with its long axis rotated to form a 45° angle with the edge 2025. Each of the sixth plurality of perforations 2820 may be a curved slit with its long axis rotated to form a 225° angle with the edge 2025. Within each row, the pattern of fluid passages 220 may be a repeating pattern of one of the fifth plurality of perforations 2815, one of the third plurality of perforations 2805, one of the sixth plurality of perforations, 2820, one of the fifth plurality of perforations 2815, one of the third plurality of perforations 2810, and one of the sixth plurality of perforations 2820, in sequence. Each alternating row of the pattern of fluid passages 220 may be shifted three positions, in either direction.

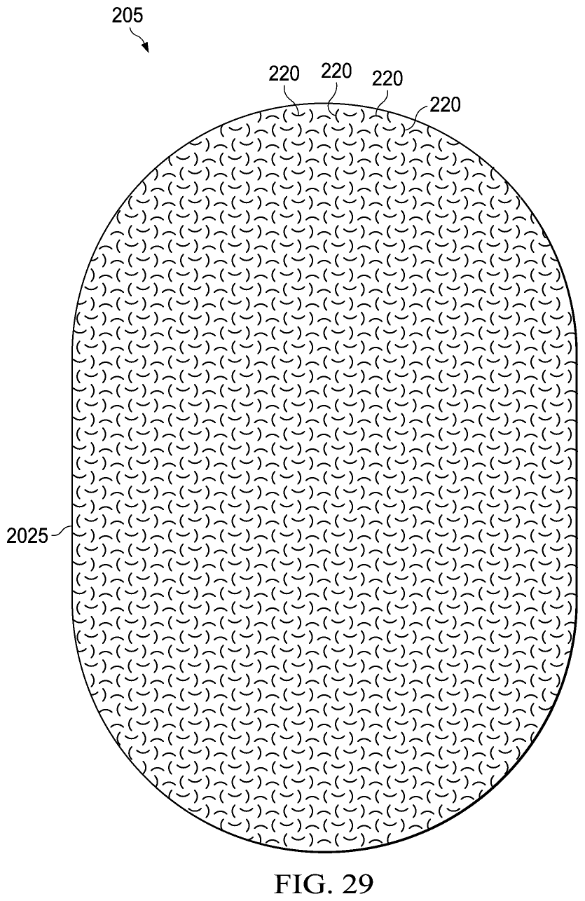

FIGS. 29 through 31 are schematic diagrams illustrating additional details that may be associated with some embodiments of the fluid passages 220. For example, as illustrated in FIG. 17, the fluid passages 220 may be distributed across the first film layer 205 in a pattern of rows. In some embodiments, each fluid passage 220 along a row may be rotated about 90° with respect to an adjacent fluid passage 220. Each fluid passage 220 along a row may be rotated about 90° clockwise or 90° counterclockwise with respect to a preceding adjacent fluid passage 220 in the row. In example embodiments of the pattern of fluid passages 220, every second row may be offset by one fluid passage 220 with respect to the previous row. The pattern of FIGS. 29 through 31 may be characterized as a pattern of offset rows. Example embodiments of the pattern of FIGS. 29 through 31 may additionally be characterized as a pattern of rotating fluid passages 22.

FIG. 29 illustrates example embodiments where the fluid passages 220 comprise curved slits. In some example embodiments, the fluid passages 220 within a row alternate between being parallel with the edge 2025 of the first film layer 205 along a long axis of the fluid passage 220 and being orthogonal to the edge 2025 of the second film layer 205 along the long axis.

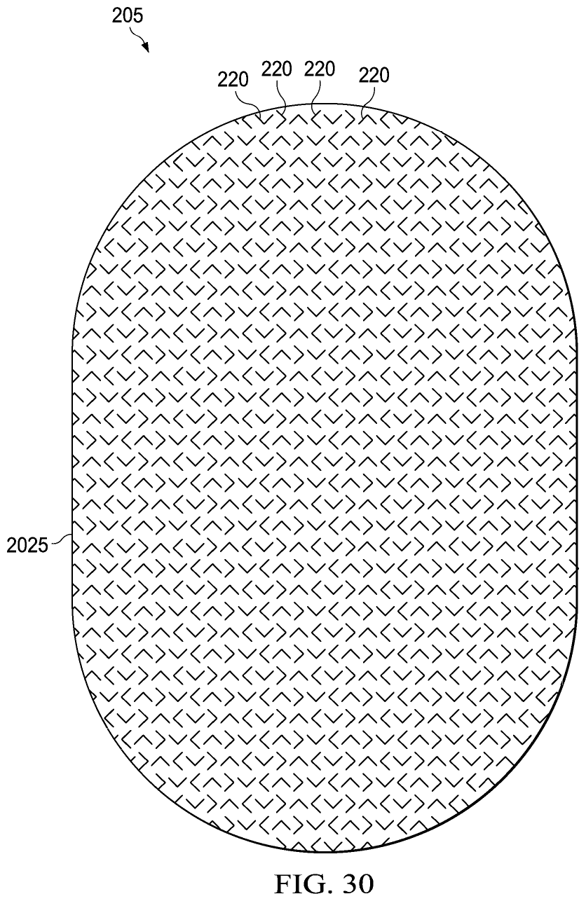

FIG. 30 shows some embodiments where the fluid passages 220 comprise chevron slits. In some example embodiments, the fluid passages 220 within a row alternate between being parallel with the edge 2025 of the first film layer 205 along a long axis of the fluid passage 220 and being orthogonal to the edge 2025 of the first film layer 205 along the long axis.

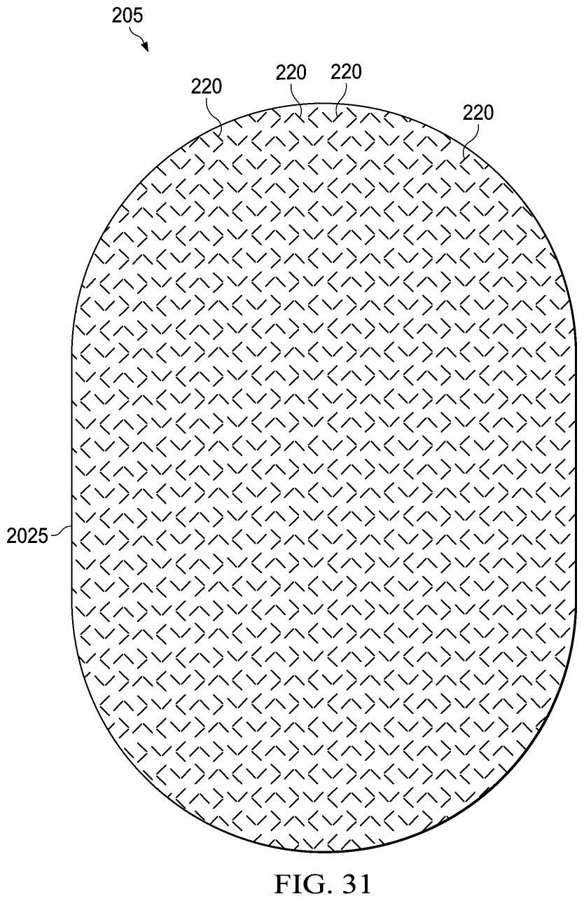

FIG. 31 further depicts illustrative embodiments where the fluid passages 220 comprise split-chevron slits. Each split-chevron slit may be formed from two orthogonal non-incidental linear slits mirrored about an axis bisecting the angle formed by the intersection of the orthogonal long axis of the linear slits. In some example embodiments, the fluid passages 220 within a row alternate between being parallel with the edge 2025 of the first film layer 205 along a long axis of the fluid passage 220 and being orthogonal to the edge 2025 of the second layer along the long axis.

In additional embodiments, P₁ may be in a range of about 4 millimeters to about 6 millimeters, P₂ may be in a range of about 3 mm to about 6 mm. In illustrative embodiments, D₁ may be in a range of about 3 mm to about 5 mm, and D₂ may be in a range of about 3 mm to 5 mm. In some embodiments, there may be an equal number of fluid passages 220 in the first plurality of perforations 2005 as the number of fluid passages 220 in the second plurality of perforations 2010.

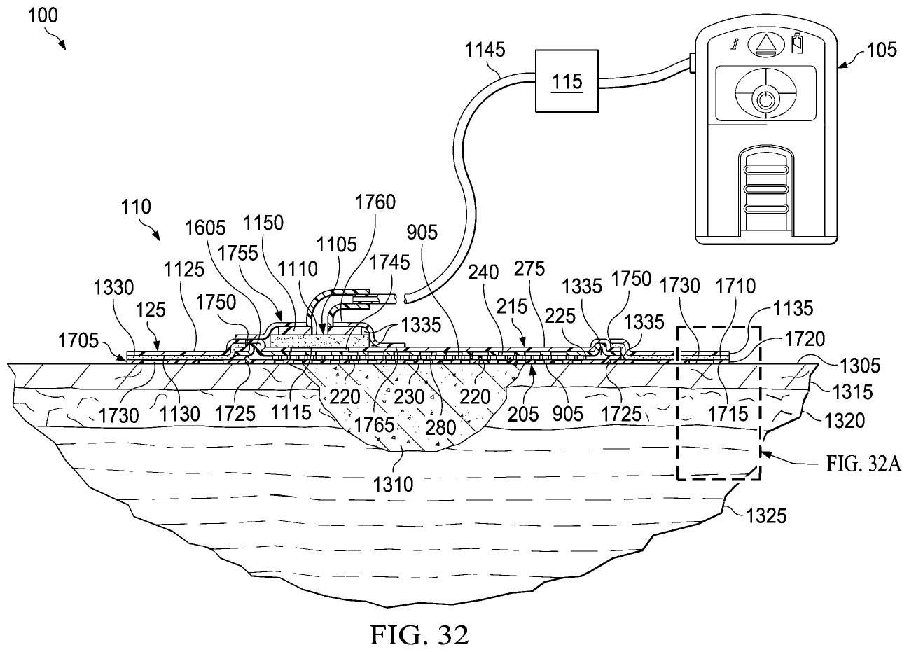

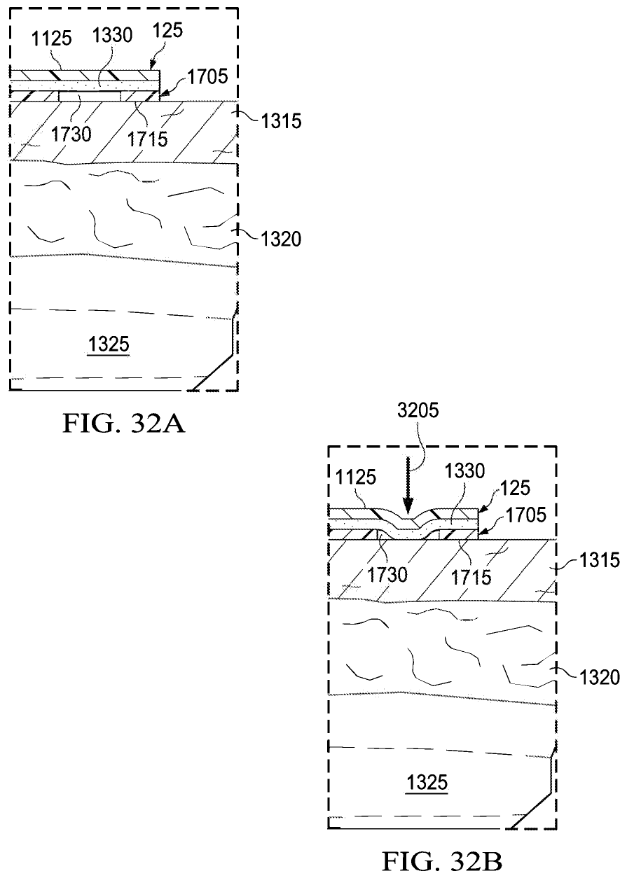

FIG. 32 is a cross-sectional view of the example dressing 110 of FIG. 17A, taken at line 32-32, applied to the example tissue site 1305, and illustrating additional details associated with the therapy system 100 of FIG. 1. In some embodiments, the dressing 110 may be applied to the tissue site 1305 and cover the wound 1310. For example, the sealing layer 1705 may be placed on a portion of the tissue site 1305 surrounding the wound 1310. At least a portion of the bottom surface 1715 of the sealing layer 1705 may be brought into contact with a portion of the epidermis 1315 surrounding the wound 1310. At least a portion of the bottom surface 230 of the first film layer 205 may be placed within, over, on, against, or otherwise proximate to the wound 1310, and a portion of the bottom surface 230 of the first film layer 205 may be coupled or adhered to a portion of the top surface 1710 of the sealing layer 1705 near the treatment aperture 1725. The cover 125, which may be coated on at least a portion of the bottom surface 1130 with adhesive 1330, may be positioned over the second film layer 215, the primary manifold 210, and the first film layer 205 such that at least a portion of the bottom surface 1130 or adhesive 1330 is brought into contact with at least a portion of the top surface 275 of the second film layer 215 and a portion of the top surface 1710 of the sealing layer 1705. The secondary manifold 1105 may be disposed over the aperture 1745 of the second film layer 215 such that at least a portion of the bottom surface 1115 of the secondary manifold 1105 is in contact with at least a portion of the top surface 275 of the second film layer 215 around the aperture 1745. The dressing interface 1150 may be disposed on at least a portion of the top surface 1110 of the secondary manifold 1105, and the connector drape 1755 may be coupled or adhered to at least a portion of a surface of the dressing interface 1150, at least a portion of a surface of the top surface 1110 of the secondary manifold 1105, at least a portion of the top surface 275 of the second film layer 215, and/or at least a portion of the top surface 1125 of the cover 125. Thus, the dressing 110 may provide the sealed therapeutic environment 1335 proximate to the wound 1310.

In operation, negative pressure may be provided to the wound 1310, and/or fluid may be removed from the wound 1310 from the sealed therapeutic environment 1335 by the negative-pressure source 105. For example, fluid may travel from the wound 1310 through at least one of the fluid passages 220, first plurality of perforations 2005, second plurality of perforations 2010, third plurality of perforations 2805, fourth plurality of perforations 2810, fifth plurality of perforations 2815, and/or sixth plurality of perforations 2820 into the portion of the sealed therapeutic environment 1335 defined by the space between the top surface 225 of the first film layer 205, the bottom surface 260 of the primary manifold 210, and the standoffs 905. Fluid may then travel through the windows 240 and into the portion of the sealed therapeutic environment 1335 defined by the space between top surface 255 of the primary manifold 210, the bottom surface 280 of the second film layer 215, and the pleat 1605. Fluid may then travel through the aperture 1745 and into the portion of the sealed therapeutic environment 1335 defined as the space between the top surface 275 of the second film layer 215, the bottom surface 1765 of the connector drape 1755, the surfaces of the dressing interface 1150 facing the secondary manifold 1105, and/or within the empty spaces of the secondary manifold 1105. Fluid may be removed from the dressing 110 through the dressing interface 1150, and optionally be collected within the container 115.

FIG. 32A is a detail view, taken at reference FIG. 32A in FIG. 32, illustrating details that may be associated with some example embodiments of the example dressing 110 of FIG. 32. For example, the sealing layer 1705 may be sufficiently tacky at the bottom surface 1715 to hold the dressing 110 in position relative to the epidermis 1315 and wound 1310, while also allowing the dressing 110 to be removed or repositioned without trauma to the epidermis 1315, wound 1310, and/or tissue site 1305. For example, the sealing layer 1705 may be formed of a silicone polyurethane material, which may form sealing couplings at the bottom surface 1715 with the epidermis 2110. In some embodiments, the bond strength or tackiness of the sealing couplings may have a peel adhesion or resistance to being peeled from a stainless steel material between about 0.5 N/25 mm to about 1.5 N/25 mm on stainless steel substrate at 25° C. at 50% relative humidity based on ASTM D3330. The sealing layer 1705 may achieve this bond strength after a contact time of less than 60 seconds. Tackiness may be considered a bond strength of an adhesive after a very low contact time between the adhesive and a substrate. In example embodiments, the sealing layer 1705 may have a thickness in a range of about 200 micrometers to about 1,000 micrometers. Removing the release liner 1780 may also expose the adhesive 1330 through the apertures 1730 of the sealing layer 1705. In the assembled state, the thickness of the sealing layer 1705 may create a gap between the adhesive 1330 and the epidermis 1315 through the apertures 1730 of the sealing layer 1705 such that the adhesive 1330 is not in contact with the epidermis 1315.

FIG. 32B illustrates additional details that may be associated with the detail view of FIG. 32A in some embodiments of the dressing 110 of FIG. 32. FIG. 32B illustrates the adhesive 1330 after it has been brought into contact with the epidermis 1315 by a force 3205 applied to the top surface 1125 of the cover 125 at the apertures 1730. In use, if the assembled dressing 110 is in the desired location, force 3205 may be applied to the top surface 1125 at the apertures 1730 to cause the adhesive 1330 to be pressed at least partially into contact with the epidermis 1315 to form bonding couplings. The bonding couplings may provide secure, releasable mechanical fixation of the dressing 110 to the epidermis 1315. The sealing couplings between the sealing layer 1705 and the epidermis 1315 may not be as mechanically strong as the bonding couplings between the adhesive 1330 and the epidermis 1315. The bonding couplings may anchor the dressing 110 to the epidermis 1315, inhibiting migration of the dressing 110.

In example embodiments, the primary manifold 210 may be formed as a polymer grid structure formed from a gel elastomer. For example, the grid members of the primary manifold 210 may be formed in the shape of brushes and combs, or any combination of geometric shapes. The windows 240 may be square, rectangular, circular, or any other suitable shape.

The systems, apparatuses, and methods described herein may provide significant advantages. For example, providing a dressing 110 with a substantially clear or optically transparent first film layer 205, adhesive 1330, second film layer 215, and cover 125 facilitates visualizing the wound 1310 through the windows 240. In illustrative embodiments, for example, as shown in FIG. 14B, portions of the second film layer 215 may be brought into contact with portions of the first film layer 205 when negative pressure is introduced to the sealed therapeutic environment 1335. In examples where at least a portion of the second film layer 215 are brought into contact with the first film layer 205 which may be in contact with the epidermis 1315 or wound 1310, the optical clarity of the epidermis 1315 or wound 1310 when viewed through the window 240 may be improved. Generally, a higher level of optical clarity may be achieved when the refractive index is constant through the lensing material in the viewing direction. In example embodiments where the wound 1310 is viewed through the cover 125, adhesive 1330, second film layer 215, an air gap in the window 240, and the first film layer 205, for example, as shown in FIG. 14A, then optical quality may be reduced as a result of the different refractive index of the air gap in the window 240 from the cover 125, adhesive 1330, second film layer 215, and the first film layer 205. However, where the second film layer 215 is brought into contact with the first film layer 205, the air gap may be eliminated or minimized. In example embodiments where at least portions of the second film layer 215 is in contact with the first film layer 205 and the refractive indexes of the layers is substantially the same, then high optical clarity may be achieved when viewing the wound 1310 through the window 240.

In illustrative embodiments, increasing the thickness of the first film layer 205 may reduce the stress placed on the wound 1310 by the primary nodes 245 or standoffs 905 when the system 100 is under negative pressure. For example, when therapeutic levels of negative pressure are introduced to the sealed therapeutic environment 1335, the pressure within the sealed therapeutic environment 1335 under the bottom surface 1130 of the cover 125 may be lower than the ambient atmospheric pressure outside of the dressing 110, such as adjacent the top surface 1125 of the cover 125. The resultant force from the pressure gradient draws the cover 125 towards the wound 1310, which also draws the primary manifold 210 towards the wound 1310. As a result, the primary nodes 245 or standoffs 905 may be drawn towards the wound 1310. In examples with a thicker first film layer 205, a greater portion of the stress field created by the primary node 245 being drawn towards the wound 1310 may be contained within the first film layer 205, and not transmitted to the wound 1310. The thickness of the first film layer 205 and the dimensions of the slots or slits forming the fluid passages 220 may be selected to selectively introduce a greater or smaller stress field to the wound 1310. For example, wider slots may be selected for the fluid passages 220 with a thicker first film layer 205 in order to prevent narrower slots or slits from remaining closed under the application of negative pressure. For example, slits may be suitable as fluid passages 220 in some applications where the first film layer 205 or second film layer 215 comprises a thickness of less than about 100 micrometers, and slots may be suitable as fluid passages 220 in some applications where the first film layer 205 or second film layer 215 comprises a thickness of greater than about 100 micrometers.

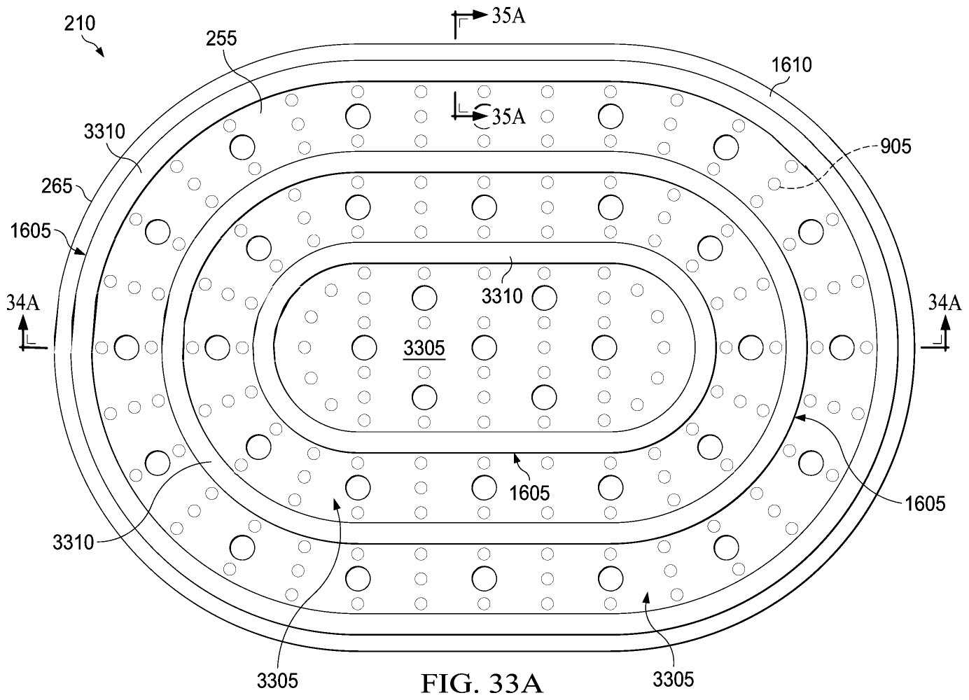

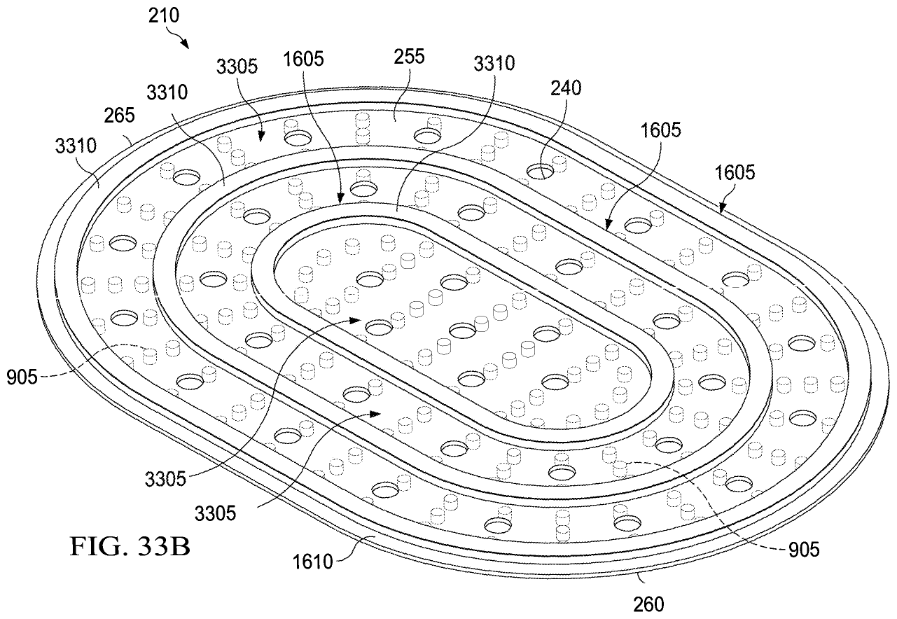

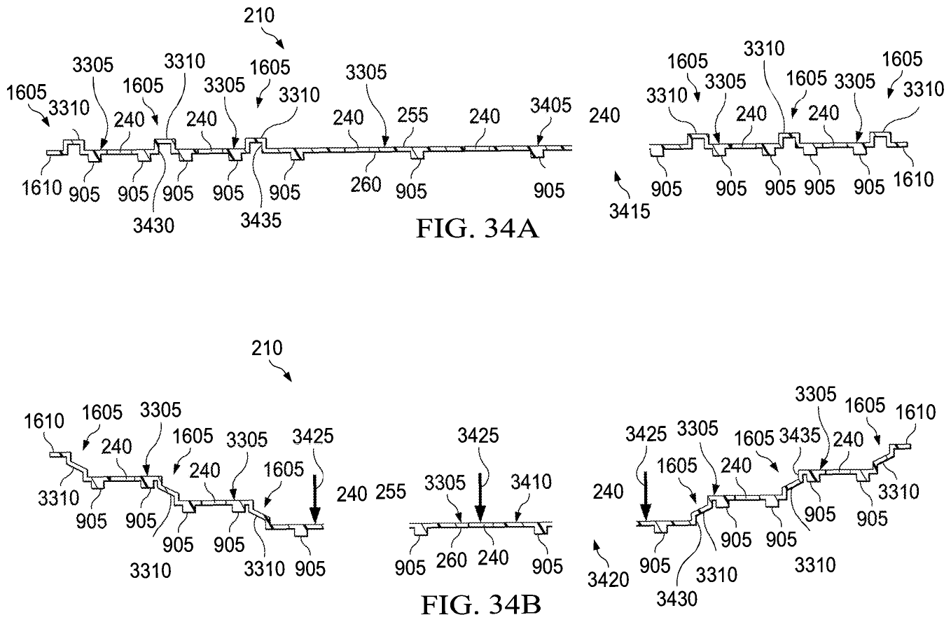

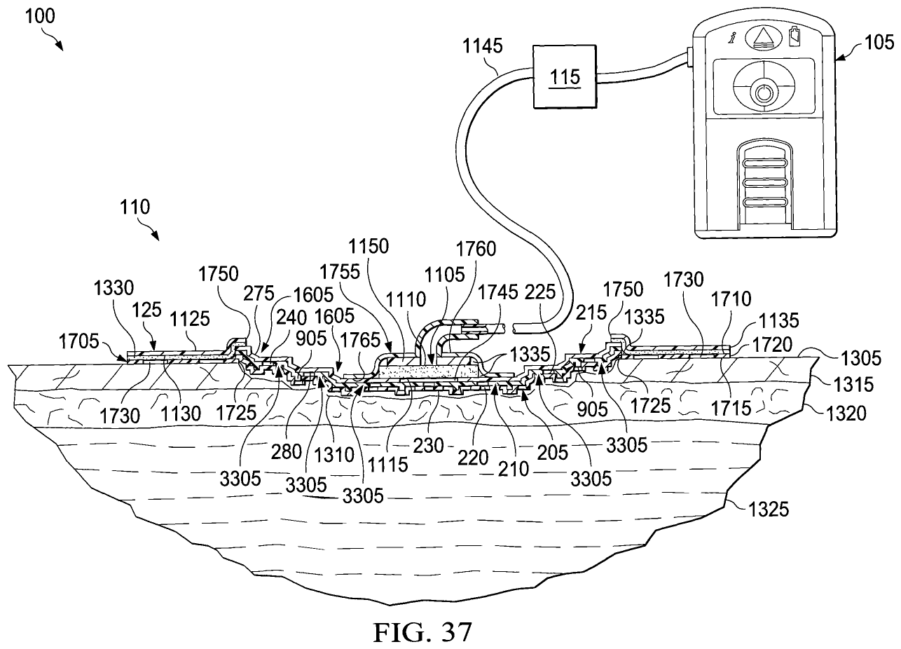

Referring to FIGS. 33A-34B, presented is another example embodiment of the primary manifold 210 suitable for use with an apparatus, dressing, and system to treat a tissue site in accordance with this disclosure. In some example embodiments, the primary manifold 210 may be configured to move between a retracted state 3405 shown in FIG. 34A and an extended state 3410 shown in FIG. 34B. The primary manifold 210 may include the top surface 255 and the bottom surface 260 positioned opposite the top surface 255 and configured to face toward a tissue site, such as the tissue site 1305 shown in FIG. 37. In some example embodiments, the primary manifold 210 may include the pleat 1605, which may be positioned adjacent to an extension zone 3305. The extension zone 3305 may be configured to extend outward from the bottom surface 260 of the primary manifold 210 toward the tissue site 1305 when the primary manifold 210 is in the extended state 3410. In the retracted state 3405 shown in FIG. 34A, the bottom surface 260 of the primary manifold 210 may have a substantially planar shape 3415 compared a shape of the bottom surface 260 in the extended state 3410.

For example, in some embodiments, the bottom surface 260 of the primary manifold 210 may be configured to form a convex shape 3420, shown in FIG. 34B, that may be positioned in conformity with the tissue site 1305 and the wound 1310, shown in FIG. 37, when the primary manifold 210 is in the extended state 3410. In some embodiments, the primary manifold 210 may be configured to move from the retracted state 3405 to the extended state 3410 upon application of negative pressure to the manifold 210, for example, when positioned at the tissue site 1305. Additionally or alternatively, in some embodiments, the primary manifold 210 may be configured to move from the retracted state 3405 to the extended state 3410 upon application of an external force 3425 to the top surface 255 and/or the bottom surface 255 of the primary manifold 210. The external force 3425 may be a pushing force on the top surface 255 and/or a pulling force on the bottom surface 260 of the primary manifold 210.

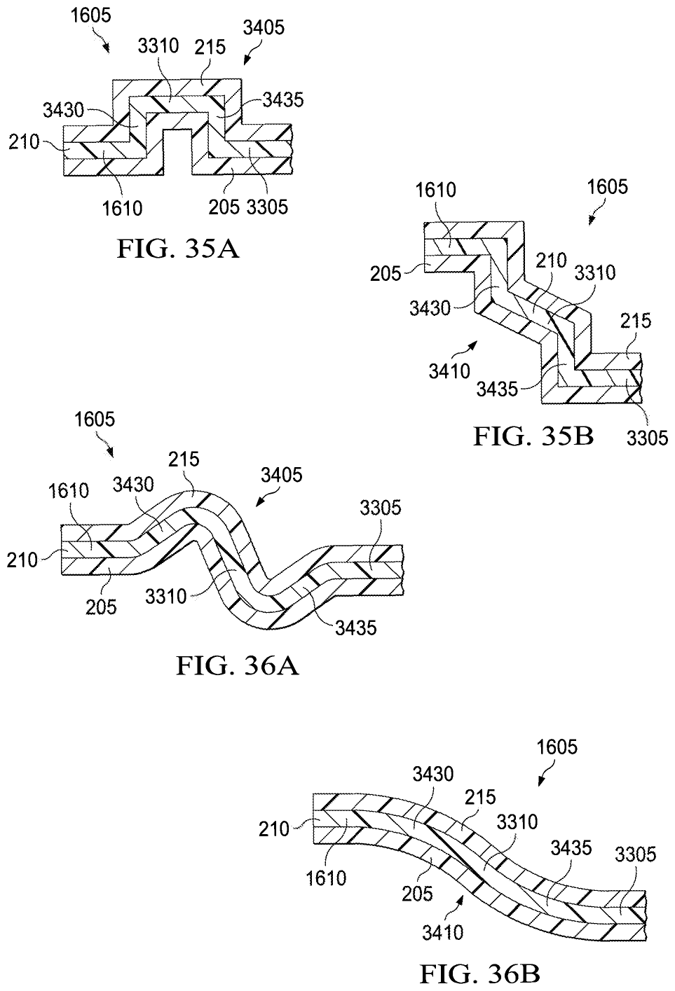

Referring to FIGS. 33A-35B, in some embodiments, the pleat 1605 may include a fold 3310 or undulation in the primary manifold 210 that is configured to permit portions of the primary manifold 210 to move away from one another, unfold, or extend when the primary manifold 210 moves from the retracted state 3405 to the extended state 3410. For example, a cross-section of the pleats 1605 shown in FIGS. 34A-35B illustrates the fold 3310 between a first portion 3430 of each of the pleats 1605 of the primary manifold 210 and a second portion 3435 of each of the pleats 1605 of the primary manifold 210. At least a portion of the first portion 3430 may be configured to overlap the second portion 3435 in the cross-section of each of the pleats 1605 when the primary manifold 210 is in the retracted state 3405. Further, at least a portion of the first portion 3430 may be configured to move away from or extend away from the second portion 3435 in the cross-section of each of the pleats 1605 when the primary manifold 210 is in the extended state 3410.

Referring more particularly to FIGS. 35A-35B, a cross-section of one embodiment the pleat 1605 is shown with the first film layer 205 and the second film layer 215 previously described in, but not limited to the embodiments of, FIGS. 2-6 and 17. As shown in FIGS. 35A-35B, the first film layer 205 and/or the second film layer 215 may be optionally coupled to and configured to move with the pleat 1605 from the retracted state 3405 to the extended state 3410.

Referring to FIGS. 36A-36B, in some example embodiments, the pleat 1605 may have the shape of an undulation or wave. For example, the fold 3310 may have the shape of a crest and a valley positioned between the first portion 3430 of the pleat 1605 and the second portion 3435 of the pleat 1605. Together, the fold 3310, the first portion 3430, and the second portion 3435 of the pleat 1605 may form the undulation or wave, which may be configured in a similar or analogous manner as described for the embodiments of FIGS. 33A-35B to permit portions of the primary manifold 210 to move away from one another, unfold, or extend when the primary manifold 210 moves from the retracted state 3405 to the extended state 3410. Further, analogous to the embodiments of FIGS. 35A-35B, the embodiments of FIGS. 36A-36B illustrate the first film layer 205 and/or the second film layer 215, which may be optionally coupled to and configured to move with the pleat 1605 from the retracted state 3405 to the extended state 3410.

Referring again to FIGS. 33A-35B, in some example embodiments, the second portion 3435 of the fold 3310 in the primary manifold 210 may be positioned between the first portion 3430 of the fold 3310 in the primary manifold 210 and the extension zone 3305. Further, in some example embodiments, the pleat 1605 may be positioned around the extension zone 3305.

Further, in some example embodiments, the primary manifold 210 may include a plurality of the pleats 1605 and a plurality of the extension zones 3305. In some examples, the plurality of pleats 1605 and the plurality of extension zones 3305 may alternate across the top surface 255 and the bottom surface 260 of the primary manifold 210. In some examples, one of the extension zones 3305 may be positioned between two of the pleats 1605 across the top surface 255 and the bottom surface 260 of the primary manifold 210. In some examples, the plurality of pleats 1605 and the plurality of extension zones 3305 may be positioned in alternating concentric rings on the top surface 255 and the bottom surface 260 of the primary manifold 210. In some examples, one or more of the plurality of pleats 1605 may be positioned circumferentially around one or more of the plurality of extension zones 3305. In some examples, one or more of the extension zones 3305 may extend outward from the bottom surface 260 of the primary manifold 210 farther than another of the extension zones 3305 when the primary manifold 210 is in the extended state 3410.

In some example embodiments, the primary manifold 210 may include or may be a polymer having a hardness in a range of about Shore 10A to about Shore 40A. Additionally or alternatively, in some examples, the primary manifold 210 may include or may be a polyurethane or silicone. Additionally or alternatively, in some examples, the primary manifold 210 may include or may be formed of a transparent material configured to provide a visual perception of the tissue site 1305 through the primary manifold 210.

Further, as previously described in, but not limited to the embodiments of, FIGS. 9A-10B and 16-17, some examples of the primary manifold 210 shown in the embodiments of FIGS. 33A-37 may include the plurality of standoffs 905, which may extend outward from one or both of the top surface 255 and/or the bottom surface 260 of the primary manifold 210. Although not shown, portions of the pleats 1605 may include surface features, such as the standoffs 905 or analogous features, configured to create a flow channel or passage on a surface of the primary manifold 210.

Further, as previously described in, but not limited to the embodiments of, FIGS. 9A-10B and 16-17, some examples of the primary manifold 210 shown in the embodiments of FIGS. 33A-37 may include the plurality of manifold openings 240 through the top surface 255 and the bottom surface 260 of the primary manifold 210. In some examples, the manifold openings 240 may be configured to provide fluid communication through the top surface 255 and the bottom surface 260 of the primary manifold 210. Further, some examples of the manifold openings 240 may be configured or positioned in a grid pattern on the primary manifold 210.

Additionally or alternatively, the manifold openings 240 may be windows configured to provide a visual perception of the tissue site 1305 through the top surface 255 and the bottom surface 260 of the primary manifold 210. Further, the primary manifold 210 in the embodiments of FIGS. 33A-37 may include the plurality of primary nodes 245 and the plurality of links 250, shown by analogy in FIG. 2, that are interconnected to define the windows. Further, in some examples, each of the primary nodes 245 may include at least one of the standoffs 905.

Further, as previously described in, but not limited to the embodiments of FIGS. 2-6 and 17, some example embodiments of the primary manifold 210 of FIGS. 33A-37 may include a polymer film adjacent to at least the bottom surface 260. The polymer film may include the plurality of fluid passages 220, 270, which may be or may include slits, slots, fenestrations or perforations as previously described. For example, the first polymer film 205 may be positioned adjacent to the bottom surface 260 of the primary manifold 210 and the second polymer film 215 may be positioned adjacent to the top surface 255 of the primary manifold 210 as shown in FIGS. 35A-36B and 37.

At least the first polymer film 205 may include the plurality of fluid passages 220. In some examples of the embodiments of FIGS. 33A-37, the first polymer film 205 may include the plurality of first fluid passages 220 and the second polymer film 215 may include the second plurality of fluid passages 270 analogous to those shown in FIGS. 2-6 and 17.

Further, analogous and without limitation to the example embodiments of FIGS. 2-6 and 17, the first polymer film 205 may have a first thickness and the second polymer film 215 may have a second thickness in the embodiments of FIGS. 33A-37. Further, the first thickness of the first polymer film 205 may be greater than the second thickness of the second polymer film 215. In some examples, the second thickness of the second polymer film 215 may be in a range of about 20 micrometers to about 500 micrometers.

Further, analogous and without limitation to the example embodiments of FIGS. 2-6 and 17, the primary manifold 210 of FIGS. 33A-37 may be bonded to at least one of the first polymer film 205 and the second polymer film 215. Further, the first polymer film 205 may be at least partially bonded to the second polymer film 215 around the primary manifold 210. Further, the primary manifold 210, the first polymer film 205, and the second polymer film 215 may each have a periphery coextensive with eachother.

Referring to FIG. 37, in some example embodiments, the therapy system 100 may include the primary manifold 210 in the examples of FIGS. 33A-36B or an apparatus including said primary manifold 210. As previously described, the therapy system 100 may include a drape configured to be positioned over at least a portion of, or form part of, the dressing 110 or other apparatus including the primary manifold 210. The drape may be configured to seal to tissue adjacent to the tissue site 1305 to form the sealed environment 1335. The drape may be one or more of the cover 125, the second film layer 215, or the connector drape 1755 as shown in FIG. 37. Although the cover 125, the second film layer 215, and the connector drape 1755 are shown as separate components, in other embodiments, the cover 125, the second film layer 215, and the connector drape 1755 may instead be a single drape or sealing structure, or a combination of one or more of the cover 125, the second film layer 215, and the connector drape 1755 configured in any suitable manner to form the sealed environment 1335. Further, one or more of the cover 125, the second film layer 215, and the connector drape 1755 may be omitted in various configurations. The therapy system 100 may further comprise a negative-pressure source 105 configured to provide negative pressure to the sealed environment 1335.

As previously described, in some example embodiments, the therapy system 100 may include the secondary manifold 1105 configured to be positioned adjacent to the primary manifold 210, or an apparatus including the primary manifold 210, opposite to the tissue site 1305. Further, in some example embodiments, the therapy system 100 may include the first polymer film 205 positioned adjacent to the bottom surface 260 of the primary manifold 210 and the second polymer film 215 positioned adjacent to the top surface 255 of the primary manifold 210. The first polymer film 205 may be configured to be positioned adjacent to the tissue site 1305, and the secondary manifold 1105 may be configured to be positioned adjacent to the second polymer film 215. In some embodiments, a portion of the drape, such as, for example, the connector drape 1755, may be configured to be positioned adjacent to a portion of the second polymer film 215. Further, in some embodiments, the secondary manifold 1105 may be configured to be positioned between the drape and the second polymer film 215.

Further, in some example embodiments, a method of treating the tissue site 1305 with negative pressure may include positioning the primary manifold 210, or an apparatus including the primary manifold 210, proximate to the tissue site 1305; applying negative pressure to the sealed environment 1335 at the tissue site 1305 including the primary manifold 210; and moving the primary manifold 210 to the extended state 3410 by operation of the negative pressure such that the bottom surface 260 of the primary manifold 210 is configured to form the convex shape 3420 in conformity with the tissue site 1305 when the primary manifold 210 is in the extended state 3410.

Further, in some example embodiments, a method of treating the tissue site 1305 with negative pressure may include positioning the primary manifold 210, or an apparatus including the primary manifold 210, proximate to the tissue site 1305; applying negative pressure to the sealed environment 1335 at the tissue site 1305 including the primary manifold 210; and extending one or more extension zones 3305 outward from the bottom surface 260 of the primary manifold 210 toward the tissue site 1305.

Further, in some example embodiments, a method of treating the tissue site 1305 according to this disclosure may include observing the tissue site 1305 through one or more openings 240 disposed through the primary manifold 210. Alternatively or additionally, some example methods for treating the tissue site 1305 according to this disclosure may include observing the tissue site 1305 through a transparent material forming at least a portion of the primary manifold 210.

In some embodiments, one or more components of the primary manifold 210, or the dressing 110 or apparatus including the primary manifold 210, may be subjected to a thermoforming process. For example, one or more of the drape, the cover 125, the primary manifold 210, the first film layer 205, and the second film layer 215 may be subjected to thermoforming to impart the previously described features and configurations to the primary manifold 210 and the dressing 110 or apparatus, such as and without limitation to, one or more of the pleats 1605, the extension zones 3305, and the standoffs 905.

In some embodiments, two or more components of the dressing 110 may be coupled together prior to the thermoforming. Additionally or alternatively, in some embodiments, two or more components of the dressing 110 may be thermoformed apart from each other and then coupled together after the thermoforming. For example, in some embodiments, the cover 125, the primary manifold 210, the first film layer 205, and the second film layer 215, may be first coupled together and then subjected to thermoforming. Alternatively, in some embodiments, two or more of the cover 125, the primary manifold 210, the first film layer 205, and the second film layer 215 may be thermoformed prior to being coupled to an adjacent component of the dressing 110.

In some embodiments, the thermoforming process includes heating a precursor material to a temperature at which the precursor material becomes pliable. In various embodiments, the parameters associated with heating the precursor material may be selected based upon factors including the material being thermoformed.

Additionally, in some embodiments, the heated precursor material may be conformed to a form or mold, for example, a mandrel. Generally, the form to which the heated precursor material is conformed may be selected based upon the desired characteristics of the resultant component of the tissue interface 120 such as the primary manifold 210. For example, the form may include a three-dimensional shape such as a portion of an interior or exterior surface of a sphere, an ellipsoid, a torus, a cylinder, a paraboloid, a hyperboloid, a cone, a prism, a pyramid, a tetrahedron, or combinations thereof. The heated precursor material may be conformed by any suitable methodology. For example, in some embodiments, the heated precursor material may be conformed to the form or mold by a vacuum. Additionally, in some embodiments, the heated precursor material may be cooled while conformed to the form and, upon cooling, one or more surface features may be imparted to one or more of the cover 125, the primary manifold 210, the first film layer 205, and the second film layer 215.

In some embodiments, in addition or as an alternative to imparting one or more surface features to a component of the dressing 110, the thermoforming process may also be effective to modify one or more parameters associated with the dressing 110, the tissue interface 120, or one or more components thereof, for example, the cover 125, the primary manifold 210, the first film layer 205, and the second film layer 215. For example, in some embodiments, the dressing 110, one or more components of the dressing 110, or some combination of the components of the dressing 110 may be characterized as exhibiting a decrease in tensile strength after the thermoforming process and/or as a result of the thermoforming process. For example, the dressing 110, one or more components of the dressing 110, or some combination of the components of the dressing 110 may be characterized as exhibiting a decrease in tensile strength in comparison to an otherwise similar dressing that has not been thermoformed. In some embodiments, the dressing 110, one or more components of the dressing 110, or some combination of the components of the dressing 110 may exhibit a decrease in tensile strength of at least 10% as a result of the thermoforming or in comparison to an otherwise similar dressing that has not been thermoformed, or a decrease in tensile strength of at least 15%, or a decrease in tensile strength of at least 20%, or a decrease in tensile strength of at least 25%, or a decrease in tensile strength of at least 30%, or a decrease in tensile strength of at least 35%, or a decrease in tensile strength of at least 40%.

Additionally or alternatively, in some embodiments, the dressing 110, one or more components of the dressing 110, or some combination of the components of the dressing 110 may be characterized as exhibiting increased flexure after the thermoforming process and/or as a result of the thermoforming process. For example, the dressing 110, one or more components of the dressing 110, or some combination of the components of the dressing 110 may be characterized as exhibiting increased flexure in comparison to an otherwise similar dressing that has not been thermoformed.

Additionally or alternatively, in some embodiments, the dressing 110, one or more components of the dressing 110, or some combination of the components of the dressing 110 may be characterized as exhibiting improved conformability with respect to a tissue site after the thermoforming process and/or as a result of the thermoforming process. For example, the dressing 110, one or more components of the dressing 110, or some combination of the components of the dressing 110 may be characterized as exhibiting improved conformability with respect to a tissue site in comparison to an otherwise similar dressing that has not been thermoformed.

For example, the increased flexure and/or the improved conformability may result from a decrease in tensile strength of the dressing 110, one or more components or features of the dressing 110, or some combination of the components of the dressing 110. Referring to FIG. 37, a cutaway view of an embodiment of the dressing 110 of FIG. 17 is illustrated positioned relative to the tissue site 1305 of a patient. As shown illustrated by FIG. 37, when positioned relative to the tissue site 1305, the dressing 110 may extend over the tissue site 1305 such that the dressing 110 is supported about its periphery by peripheral tissue. In some embodiments, the application of an external force 3425, as described, for example, in association with FIG. 34B, applied to the dressing 110 in the direction of the tissue site 1305 may cause a region of the dressing 110, one or more components of the dressing 110, or some combination of the components of the dressing 110 to experience tension. In some embodiments, a decrease in the tensile strength of the dressing 110, one or more components of the dressing 110, or some combination of the components of the dressing 110, as may result from the thermoforming process, may cause the dressing 110, one or more components of the dressing 110, or some combination of the components of the dressing 110 to exhibit increased flexure and/or improved conformability.

In some embodiments, the dressing 110 may be subjected to the thermoforming process in its entirety, for example, such that the entirety of various components of the dressing 110 may be subjected to the thermoforming process. Alternatively, in some embodiments, less than the entirety of the dressing 110 may be subjected to the thermoforming process. For example, in some embodiments, less than the entirety of one or more components of the dressing 110 may be subjected to the thermoforming process. Additionally or alternatively, in some embodiments, the dressing 110 and/or one or more components of the dressing 110 may include various regions having been subjected to varying degrees of the thermoforming process, for example, such that the dressing 110 and/or one or more components of the dressing 110 may exhibit variations in tensile strength, flexure, and/or conformability at various regions thereof. For example, in some embodiments, the dressing 110 and/or one or more components of the dressing 110 may include one or more thermoformed regions, for example, one or more tension-relief regions. In the embodiments of FIGS. 33A-37, such tension-relief regions may be configured as the pleats 1605, for example, which permit the extension zones 3305 to extend outward from the bottom surface 260 of the primary manifold 210 into conformity with the tissue site 1305 as described herein.

In some embodiments, the dressing 110 may be advantageously employed in the provision of negative-pressure therapy, for example, as a result of the decreased tensile strength, increased flexure, and/or improved conformability relative to a tissue site exhibited by the dressing 110. For example, the increased flexure and/or improved conformability of the dressing 110 may allow the dressing 110 to provide better contact between the tissue site 1305 and a tissue site-facing surface of the dressing 110. The improved contact between the dressing 110 and the tissue site 1305 may have the effect of inducing micro-strain across substantially all of the tissue site 1305, whereby cells across the tissue site 1305 experience strain, improving the outcome of the negative-pressure therapy.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, tissue interface 120, cover 125, or any combination of components may be eliminated or separated from other components for manufacture or sale. In other example configurations, the controller 130 may also be manufactured, configured, assembled, or sold independently of other components. Further features, elements, and aspects described in the context of some embodiments may also be omitted, combined, or replaced by alternative features serving the same, equivalent, or similar purpose without departing from the scope of the invention defined by the appended claims.

1. An apparatus for treating a tissue site with negative pressure, the apparatus comprising:
a primary manifold configured to move between a retracted state and an extended state, the primary manifold comprising:
a top surface and a bottom surface positioned opposite the top surface and configured to face toward the tissue site, and
a pleat positioned adjacent to an extension zone, wherein the extension zone is configured to extend outward from the bottom surface toward the tissue site when the primary manifold is in the extended state.

2. The apparatus of claim 1, wherein the bottom surface of the primary manifold is configured to form a convex shape in conformity with the tissue site when the primary manifold is in the extended state.

3.-4. (canceled)

5. The apparatus of claim 1, wherein the pleat comprises a fold in the primary manifold.

6. The apparatus of claim 1, wherein a cross-section of the pleat comprises a fold between a first portion of the primary manifold and a second portion of the primary manifold, wherein at least a portion of the first portion is configured to overlap the second portion when the primary manifold is in the retracted state, and wherein at least a portion of the first portion is configured to move away from the second portion in the extended state.

7. The apparatus of claim 6, wherein the second portion of the primary manifold is positioned between the first portion of the primary manifold and the extension zone.

8. The apparatus of claim 1, wherein the pleat is positioned around the extension zone.

9. (canceled)

10. The apparatus of claim 1, wherein the pleat comprises a plurality of pleats, wherein the extension zone comprises a plurality of extension zones, and wherein one of the extension zones is positioned between two of the pleats across the top surface and the bottom surface of the primary manifold.

11. The apparatus of claim 1, wherein the pleat comprises a plurality of pleats, wherein the extension zone comprises a plurality of extension zones, and wherein the plurality of pleats and the plurality of extension zones are positioned in alternating concentric rings on the top surface and the bottom surface of the primary manifold.

12. (canceled)

13. The apparatus of claim 1, wherein the pleat comprises a plurality of pleats, wherein the extension zone comprises a plurality of extension zones, and wherein one or more of the extension zones extends outward from the bottom surface of the primary manifold farther than another of the extension zones when the primary manifold is in the extended state.

14. The apparatus of claim 1, further comprising a plurality of manifold openings through the top surface and the bottom surface, wherein the manifold openings are configured to provide fluid communication through the top surface and the bottom surface of the primary manifold.

15. (canceled)

16. The apparatus of claim 1, wherein the primary manifold further comprises a plurality of standoffs extending outward from one or both of the top surface and the bottom surface.

17. The apparatus of claim 1, further comprising a first polymer film and a second polymer film, the first polymer film positioned adjacent to the bottom surface of the primary manifold and the second polymer film positioned adjacent to the top surface of the primary manifold, wherein at least the first polymer film includes a plurality of fluid passages.

18. A system for treating a tissue site with negative pressure, comprising:
the apparatus of claim 1;
a drape configured to be positioned over at least a portion of the apparatus and seal to tissue adjacent to the tissue site to form a sealed environment; and
a negative-pressure source configured to provide negative pressure to the sealed environment.

19. (canceled)

20. A method of treating a tissue site with negative pressure, the method comprising:
positioning the apparatus of claim 1 proximate to the tissue site;
applying negative pressure to a sealed environment at the tissue site including the apparatus; and
extending one or more extension zones outward from the bottom surface of the primary manifold toward the tissue site.

21.-24. (canceled)

25. The apparatus of claim 14, wherein the manifold openings are windows further configured to provide a visual perception of the tissue site through the top surface and the bottom surface of the primary manifold.

26.-27. (canceled)

28. The apparatus of claim 25, wherein the primary manifold further comprises a plurality of standoffs extending outward from one or both of the top surface and the bottom surface, wherein the primary manifold includes a plurality of primary nodes and a plurality of links that are interconnected to define the windows, and wherein each of the primary nodes comprises at least one of the standoffs.

29. The apparatus of claim 1, further comprising a polymer film adjacent to the bottom surface of the primary manifold, the polymer film including a plurality of fluid passages.

30. (canceled)

31. The apparatus of claim 17, wherein:
the first polymer film has a first thickness;
the second polymer film has a second thickness; and
the first thickness is greater than the second thickness.

32.-35. (canceled)

36. The apparatus of claim 17, wherein the plurality of fluid passages comprise a plurality of slots or slits, each of the slots or slits having a length less than 5 millimeters and a width less than 2 millimeters.

37. (canceled)

38. The system of claim 18, further comprising a secondary manifold configured to be positioned adjacent the apparatus opposite the tissue site.

39. The system of claim 38, further comprising a first polymer film positioned adjacent to a bottom surface of the primary manifold and a second polymer film positioned adjacent to a top surface of the primary manifold, wherein the first polymer film is configured to be positioned adjacent to the tissue site, and wherein the secondary manifold is configured to be positioned adjacent to the second polymer film.

40. (canceled)

41. The system of claim 39, wherein the secondary manifold is configured to be positioned between the drape and the second polymer film.

42. (canceled)
